(19) United States
(12) Patent Application Publication
(10) Pub. No.: US 2025/0340667 A1
(43) Pub. Date: Nov. 6, 2025

(54) Cancer Treatment comprising an anti-MSLN/CD137 antibody and a chemotherapeutic

(71) Applicant: Merck Patent GmbH, Darmstadt (DE)

(72) Inventor: Chunxiao XU, Billerica, MA (US)

(73) Assignee: Merck Patent GmbH, Darmstadt (DE)

(21) Appl. No.: 19/106,494

(22) PCT Filed: Aug. 23, 2023
(86) PCT No.: PCT/EP2023/073075

§ 371 Date: Feb. 25, 2025

Related U.S. Application Data
(60) Provisional application No. 63/401,292, filed on Aug. 26, 2022.

(51) Int. Cl. C07K 16/28; A61K 39/00; A61K 45/06; A61P 35/00

(52) U.S. Cl. CPC C07K 16/2896; A61K 45/06; A61P 35/00; C07K 16/28; A61K 2039/505; C07K 2317/31; C07K 2317/526; C07K 2317/565

(57) ABSTRACT

The application relates to the use of an antibody molecule that binds to MSLN and CD137 and a chemotherapeutic in the treatment of cancer in a patient.

### FIELD OF THE INVENTION

The present invention relates to the use of a bispecific antibody molecule that binds to MSLN and CD137 and a chemotherapeutic in the treatment of cancer in a patient.

### BACKGROUND

Costimulatory pathways, such as the CD137/4-1BB pathway, are vital in driving productive anticancer immunity, with strong genetic evidence supporting their role in mediating anticancer immune responses¹⁻⁵. Therefore, a growing number of studies aim to modify signals through the use of agonistic antibodies targeting costimulatory molecules to boost antitumor T cell responses.

CD137 (also known as 4-1BB or TNFRSF9) is an inducible T cell surface receptor belonging to the tumor necrosis factor receptor (TNFR) superfamily, which activates diverse cellular functions, including production of type 1 interferons and modulation of antigen-activated T cell survival⁶. CD137 is expressed on the surface of activated CD4⁺ and CD8⁺ T cells, monocytes, and B lymphocytes. The expression of CD137 can be induced via T cell receptor (TCR) stimulation⁷, which is termed “signal 1” (TCR/CD3/MHC interaction between human T cell and target cell). Activation of the CD137 pathway promotes T cell differentiation and survival⁸⁻¹⁰, provides strong protection against activation-induced T cell death, and increases cytotoxicity¹¹⁻¹³.

The efficacy of anti-CD137 therapy has been demonstrated in multiple nonclinical tumor models¹⁴⁻¹⁸. Anti-CD137 agonistic antibodies have been shown to induce effector molecule release from CD8⁺ T cells, increase proliferation, and prevent cytotoxic T lymphocyte (CTL) anergy, thereby breaking T cell tolerance towards tumor antigens¹⁹ and increasing persistence of tumor-specific T cells²⁰. Based on promising nonclinical antitumor effects, two 1^{st} generation CD137 agonists, utomilumab (PF-05082566) and urelumab (BMS-663513), have been developed and investigated clinically. Clinical studies of both utomilumab and urelumab monotherapy were however suspended, due to low efficacy of utomilumab and hepatotoxicity of urelumab^{21,22}. Further structure analysis indicated that these outcomes were mediated by a recognized epitope on CD137 and by Fc gamma receptor (FcγR) ligand-dependent clustering²³.

To overcome either low antitumor efficacy or hepatotoxicity mediated by FcγR ligand-dependent clustering of the 1^{st} generation CD137 agonists, strategies that deliver CD137 agonists to the tumor site are required to reduce systemic toxicities while allowing for clinical administration²⁴. These 2^{nd} generation CD137 agonists are either monospecific antibodies claiming to bind CD137 epitopes that are not associated with liver toxicity or are CD137/tumor associated antigen (TAA) bispecific antibodies that are targeted to the tumor microenvironment (TME), do not bind FcγRs, and are linked to antibodies targeting tumor antigens or tumor tissue^{15,25,26}.

Mesothelin (MSLN) is a 40-kilodalton (kD) membrane-bound protein that is overexpressed in various cancers, including mesothelioma, ovarian cancer, lung cancer, and pancreatic cancer²⁷⁻³⁸. The limited expression of MSLN on normal human tissues and its high expression in many common cancers make it an attractive candidate for cancer therapy. Several agents are in various stages of development to treat patients with MSLN-expressing tumors, including a monoclonal antibody, immunotoxin, tumor vaccine, and an antibody drug conjugate³⁹.

M9657 (FS22-172-003-AA/FS28-256-271 of WO 2020/011976) is a first-in-class, tumor-targeted conditional agonist antibody developed to enhance antitumor immune responses in the TME. The bispecific antibody M9657 was engineered in a tetravalent bispecific antibodies (mAb²) format, with the Fab portion binding to the tumor antigen MSLN and a modified CH3 domain as the Fc antigen binding (Fcab) portion binding to CD137. M9657 has a human IgG1-LALA backbone, which blocks binding to Fcγ receptors but retains FcRn binding for IgG-like pharmacokinetics (PK). High expression of MSLN on tumor cells should result in increased binding and crosslinking of the antibody molecules and interaction with the CD137 trimer, consequently increasing CD137 agonism. Therefore, the clustered M9657 may function as a bridge to link the CD137 trimer and tumor cells. As M9567 promotes CD137 activation signaling within the TME, which avoids systemic immune activation, it is expected that M9657 will provide advantages over monospecific CD137 antibodies. In preclinical studies, M9657 displayed MSLN target-dependent and dose-dependent anti-tumor immunity.

Nonetheless, there remains a need in the art for additional anti-cancer therapies.

### STATEMENTS OF THE INVENTION

Accumulating evidence indicates that chemotherapeutic drugs can induce immunogenic death of tumor cells, which releases or exposes these immunogenic tumor antigens, allowing for their interaction with innate immune cells such as monocytes, macrophages, and dendritic cells (DCs)^{40,41}. This leads to activation and maturation of these immune cells, which migrate to draining lymph nodes loaded with cancer-derived antigen-specific cargo. Cancer antigens are then presented to T cells, which enable a potent anticancer adaptive immune response. Conventional chemotherapeutic agents induce immunogenic cell death by interfering directly with DNA or targeting key proteins required for cell division⁴². Immunogenic dead tumor cells can release tumor-associated antigens (TAAs) and danger-associated molecular patterns (DAMPs), both of which recruit immune cells in TME positively⁴³. Some chemotherapeutic agents have been reported to deplete myeloid-derived suppressor cells (MDSC), cancer-associated neutrophils, and macrophages^{44, 45}. Optimal doses of some chemotherapeutic agents can promote effector T cell proliferation and Treg depletion⁴⁶. The optimal integration of immunotherapy with standard of care (SOC) chemotherapy to achieve additive or synergistic clinical activity is being actively investigated in both nonclinical and clinical trials.

As described above, chemotherapy can facilitate the use of immunotherapy in the treatment of cancer. However, immune cell activation would not be limited to the TME in which an increased cancer antigen load arises, raising concerns regarding its efficacy and specificity. Particularly CD137 agonist molecules have in the past been held back due to concerns with regards to liver inflammation and clinical efficacy The present inventors recognized that target-specific anti-tumor activity could be enhanced by combining MSLN expression-dependent CD137 co-stimulation of T cells with chemotherapy. Surprisingly, the present inventors were able to show that the combination of an antibody molecule that binds MSLN and CD137 and a chemotherapeutic resulted in greater anti-tumor effect in vivo in mouse tumor models than the combined increase in anti-tumor effect observed when mice were treated with either the antibody molecule that binds MSLN and CD137 or the chemotherapeutic alone. In other words, the anti-tumor effect of the combination treatment was not just additive but synergistic. This was unexpected. The effect achieved by a combination of two agents is synergistic if the effect is greater than the total of the individual effects of the two agents combined⁴⁷. Accordingly, the present inventors found that the combination of an antibody molecule that binds MLSN and CD137 and a chemotherapeutic increased the anti-tumor effect in vivo in mouse tumor models in a synergistic manner. A similar synergistic anti-tumor effect is expected when human patients are treated with a combination of an antibody molecule that binds MSLN and CD137 and a chemotherapeutic.

Due to the lack of cross-reactivity between M9657 (SEQ ID NO: 2 and SEQ ID NO: 10) and mouse MSLN and CD137 proteins, anti-mMSLN-mCD137-huIgG1-LALA (FS122m) (SEQ ID NO: 84 and SEQ ID NO: 85) was developed, a surrogate antibody of M9657 for in vivo studies in mouse tumor models. As with M9657, FS122m was engineered in a tetravalent bispecific antibody (mAb²) format with the Fab portion targeted to bind to the tumor antigen mouse MSLN and a modified CH3 domain as an Fcab portion targeted to murine CD137. FS122m has a human IgG1 backbone with LALA mutations to abrogate the binding to Fcγ receptor. The binding affinity of FS122m for mouse MSLN/CD137 is similar to the binding affinity of M9657 for human MSLN/CD137.

As already summarized above, the present inventors showed that the combination of FS122m and either of the two chemotherapeutics cisplatin or gemcitabine was capable of retarding tumor growth or reducing tumor volume in ST26 and JC mouse tumor models to a greater extent than the combined tumor growth retardation or tumor volume reduction observed when mice were treated with either FS122m or with cisplatin or gemcitabine alone. The present inventors also showed that combined treatment with FS122m and either cisplatin or gemcitabine increased median survival and increased the percentage of mice with complete tumor regression in the same mouse tumor models compared with the combined increase in median survival and percentage of mice with complete tumor regression observed when mice were treated with either FS122m or with cisplatin or gemcitabine alone. The present inventors thus showed that the combination of FS122m and either cisplatin or gemcitabine enhanced anti-tumor activity, as measured by tumor growth retardation/tumor volume reduction, median survival, and the percentage of mice showing complete tumor regression in ST26 and JC mouse tumor models, in a synergistic manner.

These nonclinical studies in mouse tumor models support the expectation that the combination of an antibody molecule that binds MSLN and CD137 and a chemotherapeutic enhances anti-tumor activity and in a synergistic manner in human patients. These findings present combination therapy with an antibody molecule that binds MSLN and CD137 and a chemotherapeutic as a new therapeutic strategy to improve cancer treatment.

The present invention thus provides an antibody molecule that binds MSLN and CD137 for use in a method of treating cancer in a patient, wherein the method comprises administering the antibody in combination with a chemotherapeutic. The present invention also provides a chemotherapeutic for use in a method of treating cancer in a patient, wherein the method comprises administering the chemotherapeutic in combination with an antibody molecule that binds MSLN and CD137.

The antibody molecule that binds MSLN and CD137 may be an immunoglobulin or an antigen-binding fragment thereof. For example, the antibody molecule may be an IgG, IgA, IgE or IgM molecule, preferably an IgG molecule, such as an IgG1, IgG2, IgG3 or IgG4 molecule, more preferably an IgG1 or IgG2 molecule, most preferably an IgG1 molecule, or a fragment thereof. In a preferred embodiment, the antibody molecule is a complete immunoglobulin molecule.

The antibody molecule may comprise at least one, preferably more than one, complementary determining region (CDR)-based binding site for MSLN and at least one, preferably more than one, binding site for CD137 in a constant domain of the bispecific antibody molecule, preferably in the CH3 domain.

The binding site for CD137 may comprise a first sequence and a second sequence located in the AB and EF structural loops of the CH3 domain of the antibody molecule. Preferably, the first sequence has the sequence set forth in SEQ ID NO: 87. Preferably, the second sequence has the sequence set forth in SEQ ID NO: 88. More preferably, the first sequence has the sequence set forth in SEQ ID NO: 87 and the second sequence has the sequence set forth in SEQ ID NO: 88. According to the IMGT numbering scheme, the first sequence may be located between positions 14 and 17 of the CH3 domain of the antibody molecule. The second sequence may be located between positions 91 and 99 of the CH3 domain of the antibody molecule according to the IMGT numbering scheme. Preferably, the sequence of the CH3 domain of the antibody molecule has the sequence set forth in SEQ ID NO: 86.

In a preferred embodiment, the bispecific antibody molecule comprises a CH3 domain which comprises, has, or consists of the CH3 domain sequence of FS22-172-003 set forth in SEQ ID NO: 86. The CH3 domain of the bispecific antibody molecule may optionally comprise an additional lysine residue (K) at the immediate C-terminus of the CH3 domain sequence.

A number of Fabs that bind MSLN are known from WO 2020/011976. The complementary determining region (CDR)-based binding site for MSLN can comprise CDRs 1-6 of any of these Fabs. The antibody molecule that binds MSLN and CD137 may therefore comprise CDRs 1-6 set forth in SEQ ID NOs 4, 6, 8, 12, 14, and 16 [FS28-256-271]; SEQ ID NOs 20, 22, 24, 12, 14 and 28 [FS28-024-052]; SEQ ID NOs 4, 6, 8, 12, 14 and 34 [FS28-256-021]; SEQ ID NOs 4, 6, 8, 12, 14, and 39 [FS28-256-012]; SEQ ID NOS 43, 6, 45, 12, 14 and 34 [FS28-256-023]; SEQ ID NOs 4, 6, 8, 12, 14 and 49 [FS28-256-024]; SEQ ID NOs 43, 6, 45, 12, 14 and 49 [FS28-256-026]; SEQ ID NOs 4, 6, 8, 12, 14 and 16 [FS28-256-027]; SEQ ID NOs 53, 6, 55, 12, 14 and 34 [FS28-256-001]; SEQ ID NOs 53, 6, 55, 12, 14 and 49 [FS28-256-005]; SEQ ID NOs 60, 6, 62, 12, 14 and 39 [FS28-256-014]; SEQ ID NOs 43, 6, 45, 12, 14 and 39 [FS28-256-018]; SEQ ID NOs 67, 6, 55, 12, 14 and 39 [FS28-256]; SEQ ID NOs 21, 23, 72, 12, 14 and 28 [FS28-024-051]; SEQ ID NOs 21, 23, 77, 12, 14 and 28 [FS28-024-053]; or SEQ ID NOs 21, 23, 82, 12, 14 and 28 [FS28-024].

A number of bispecific antibody molecules that binds MSLN and CD137 are known from WO 2020/011976. Antibody M9657 of this application is identical to antibody FS22-172-003-AA/FS28-256-271 of WO 2020/011976. Any of these antibodies can be used and are hereby incorporated by reference. The antibody molecule that binds MSLN and CD137 may therefore comprise the heavy chain and light chain set forth in SEQ ID NOs 2 and 10 (FS22-172-003-AA/FS28-256-271), SEQ ID NOs 18 and 26 (FS22-172-003-AA/FS28-024-052), SEQ ID NOs 30 and 32 (FS22-172-003-AA/FS28-256-021), SEQ ID NOs 36 and 37 (FS22-172-003-AA/FS28-256-012), SEQ ID NOs 41 and 32 (FS22-172-003-AA/FS28-256-023), SEQ ID NOs 30 and 47 (FS22-172-003-AA/FS28-256-024), SEQ ID NOs 41 and 47 (FS22-172-003-AA/FS28-256-026), SEQ ID NOs 30 and 10 (FS22-172-003-AA/FS28-256-027), SEQ ID NOS 51 and 32 (FS22-172-003-AA/FS28-256-001), SEQ ID NOs 51 and 47 (FS22-172-003-AA/FS28-256-005), SEQ ID NOs 58 and 37 (FS22-172-003-AA/FS28-256-014), SEQ ID NOs 41 and 37 (FS22-172-003-AA/FS28-256-018), SEQ ID NOs 65 and 37 (FS22-172-003-AA/FS28-256), SEQ ID NOs 70 and 26 (FS22-172-003-AA/FS28-024-051), SEQ ID NOs 75 and 26 (FS22-172-003-AA/FS28-024-053), or SEQ ID NOs 80 and 26 (FS22-172-003-AA/FS28-024), respectively. Preferably, the antibody molecule that binds MSLN and CD137 comprises the heavy chain sequence set forth in SEQ ID NO: 2 and the light chain sequence set forth in SEQ ID NO: 10 (FS22-172-003-AA/FS28-256-271).

The chemotherapeutic may be an alkylating agent or an antimetabolite. Preferably, the antimetabolite is selected from the group including azacitidine, 5-fluorouracil (5-FU), 6-mercaptopurine (6-MP), capecitabine (Xeloda), cladribine, clofarabine, cytarabine (Ara-C), decitabine, floxuridine, fludarabine, gemcitabine (Gemzar), hydroxyurea, methotrexate, nelarabine, pemetrexed (Alimta), pentostatin, pralatrexate, thioguanine, and a trifluridine/tipiracil combination. More preferably, the antimetabolite may be gemcitabine.

The alkylating agent is preferably selected from the group including altretamine, bendamustine, busulfan, carboplatin, carmustine, chlorambucil, cisplatin, cyclophosphamide (CPA), dacarbazine, ifosfamide, lomustine, mechlorethamine, melphalan, oxaliplatin, temozolomide, thiotepa, and trabectedin. More preferably, the alkylating agent may be cisplatin.

The membrane-bound protein mesothelin (MSLN) has been shown to be expressed in several cancers. All of ovarian cancer, pancreatic adenocarcinoma, mesothelioma and non-small cell lung carcinomas have been shown to express high levels of MSLN. The present inventors found that this is also the case for cervical carcinoma. Without wishing to be bound by theory, it is thought that binding of the antibody molecule to MSLN is expected to result in antibody crosslinking, binding to CD137 expressed at the surface of an immune cell, followed by CD137 clustering and activation, ultimately resulting in activation of the immune cell.

Accordingly, the cancer to be treated is preferably a cancer that expresses or has been determined to express MSLN. More preferably, the cancer is selected from the group including ovarian cancer, pancreatic adenocarcinoma, mesothelioma, cervical carcinoma and non-small cell lung cancer.

Combination therapy of FS122m and cisplatin or gemcitabine resulted in statistically significantly greater anti-tumor activity in CT26 and JC mouse tumor models than the combined anti-tumor activity observed when mice were treated with either FS122m or with cisplatin or gemcitabine alone. The effect achieved by a combination of two agents is synergistic if the effect is greater than the total of the individual effects of the two agents combined⁴⁷. Combination therapy of FS122m and cisplatin or gemcitabine therefore enhanced anti-tumor activity in CT26 and JC mouse tumor models in a synergistic manner. Accordingly, in one embodiment, treatment with the antibody molecule that binds MSLN and CD137 in combination with a chemotherapeutic results in a greater anti-tumor effect than the anti-tumor effect observed when patients are treated with either the antibody molecule that binds MSLN and CD137 or the chemotherapeutic alone. Preferably, treatment with the antibody molecule that binds MSLN and CD137 in combination with a chemotherapeutic results in a greater anti-tumor effect than the combined anti-tumor effect observed when patients are treated with either the antibody molecule that binds MSLN and CD137 or the chemotherapeutic alone. The anti-tumor effect may be tumor growth inhibition or retardation, tumor volume reduction, increase in median survival, or increase in the percentage of patients experiencing complete tumor regression. Preferably, the anti-tumor effect is tumor growth inhibition or retardation, or tumor volume reduction. The anti-tumor effect may thus be tumor growth inhibition or retardation. The anti-tumor effect may be a reduction in the tumor volume. The anti-tumor effect may be an increase in median survival of the patient. The anti-tumor effect may be an increase in the percentage of patients experiencing complete tumor regression, such as a clinical complete response, or a pathological complete response. The determination of these anti-tumor effects is within the capabilities of the skilled person.

The bispecific antibody molecule that binds MSLN and CD137 and the chemotherapeutic can be administered to a subject by any suitable means. Accordingly, in one embodiment the antibody molecule that binds MSLN and CD137 and/or the chemotherapeutic is administered parenterally. The antibody molecule that binds MSLN and CD137 and/or the chemotherapeutic may be administered intravenously, intramuscularly, subcutaneously, intraperitoneally or spinally. The antibody molecule that binds MSLN and CD137 and/or the chemotherapeutic may be administered by injection or infusion.

The antibody molecule that binds MSLN and CD137 and/or the chemotherapeutic may be administered non-parenterally. The antibody molecule that binds MSLN and CD137 and/or the chemotherapeutic may be administered orally, intranasally, vaginally, rectally, sublingually, or topically.

The antibody molecule that binds MSLN and CD137 and the chemotherapeutic can be part of the same formulation or part of separate formulations, but preferably are provided as separate formulations. Accordingly, the antibody molecule that binds MSLN and CD137 and the chemotherapeutic may be administered to the patient concomitantly or sequentially, but preferably administered sequentially.

Where the antibody molecule that binds MSLN and CD137 and the chemotherapeutic are administered to the patient sequentially, they are preferably administered to the patient within 4 days of each other, more preferably within 3 days of each other, more preferably within 2 days of each other, or sequentially on the same day.

The present invention also provides a method of treating cancer comprising administering to the individual in need thereof an antibody molecule that binds MSLN and CD137 and a chemotherapeutic. Preferably, the method of treating cancer comprises administering to the individual in need thereof a therapeutically effective amount of the antibody molecule that binds MSLN and CD137 and a therapeutically effective amount of the chemotherapeutic. In one embodiment, the method may comprise determining whether a cancer in a patient expresses MSLN and treating the patient if the cancer has been determined to express MSLN. Alternatively, the method may comprise a step of ordering the results of a test determining whether a cancer in a patient expresses MSLN and treating the patient if the test results show that the cancer expresses MSLN.

The present invention also provides a use of an antibody molecule that binds MSLN and CD137 for the manufacture of a medicament for the treatment of cancer, wherein the antibody molecule that binds MSLN and CD137 is administered in combination with a chemotherapeutic. The present invention also provides a use of a chemotherapeutic for the manufacture of a medicament for the treatment of cancer, wherein the chemotherapeutic is administered in combination with an antibody molecule that binds MSLN and CD137.

The present invention also provides a kit comprising an antibody molecule that binds MSLN and CD137 and a pharmaceutically acceptable excipient and a chemotherapeutic and a pharmaceutically acceptable excipient.

Thus the present invention provides:
[1] An antibody molecule that binds MSLN and CD137 for use in a method of treating cancer in a patient, wherein the method comprises administering the antibody in combination with chemotherapeutic.
[2] A chemotherapeutic for use in a method of treating cancer in a patient, wherein the method comprises administering the chemotherapeutic in combination with an antibody molecule that binds MSLN and CD137.
[3] A method of treating cancer in an individual, the method comprising administering to the individual an antibody molecule that binds MSLN and CD137 and a chemotherapeutic.
[4] Use of an antibody molecule that binds MSLN and CD137 for the manufacture of a medicament for the treatment of cancer, wherein the antibody molecule that binds MSLN and CD137 is administered in combination with a chemotherapeutic.
[5] Use of a chemotherapeutic for the manufacture of a medicament for the treatment of cancer, wherein the chemotherapeutic is administered in combination with an antibody molecule that binds MSLN and CD137
[6] A kit comprising
   (a) an antibody molecule that binds MSLN and CD137 and a pharmaceutically acceptable excipient; and
   (b) a chemotherapeutic and a pharmaceutically acceptable excipient.
[7] The antibody molecule or chemotherapeutic for use, method, use, or kit according to [1] to [6], wherein the antibody molecule that binds MSLN and CD137 comprises
   (a) a complementary determining region (CDR)-based antigen-binding site for MSLN; and
   (b) a CD137 antigen-binding site located in a CH3 domain of the antibody molecule.
[8] The antibody molecule or chemotherapeutic for use, method, use, or kit according to [7], wherein the antibody molecule that binds MSLN and CD137 comprises two or more of
   (a) a complementary determining region (CDR)-based antigen-binding site for MSLN; and
   (b) a CD137 antigen-binding site located in a CH3 domain of the antibody molecule.
[9] The antibody molecule or chemotherapeutic for use, method, use, or kit according to [1] to [8], wherein the antibody molecule that binds MSLN and CD137 is an IgG, IgA, IgE, IgM molecule, or antigen-binding fragment thereof.
[10] The antibody molecule or chemotherapeutic for use, method, use, or kit according to [9], wherein the antibody molecule that binds MSLN and CD137 is an IgG molecule, or antigen-binding fragment thereof.
[11] The antibody molecule or chemotherapeutic for use, method, use, or kit according to [10], wherein the antibody molecule that binds MSLN and CD137 is an IgG1 or IgG2 molecule, or antigen-binding fragment thereof.
[12] The antibody molecule or chemotherapeutic for use, method, use, or kit according to [11], wherein the antibody molecule that binds MSLN and CD137 is an IgG1 molecule, or antigen-binding fragment thereof.
[13] The antibody molecule or chemotherapeutic for use, method, use, or kit according to [8] to [12], wherein the CDR-based antigen-binding site for MSLN comprises CDRs 1-6 set forth in:
   (i) SEQ ID NOs 4, 6, 8, 12, 14, and 16, respectively [FS28-256-271];
   (ii) SEQ ID NOs 20, 22, 24, 12, 14 and 28, respectively [FS28-024-052];
   (iii) SEQ ID NOs 4, 6, 8, 12, 14 and 34, respectively [FS28-256-021];
   (iv) SEQ ID NOs 4, 6, 8, 12, 14, and 39, respectively [FS28-256-012];
   (v) SEQ ID NOs 43, 6, 45, 12, 14 and 34, respectively [FS28-256-023];
   (vi) SEQ ID NOs 4, 6, 8, 12, 14 and 49, respectively [FS28-256-024];
   (vii) SEQ ID NOs 43, 6, 45, 12, 14 and 49, respectively [FS28-256-026];
   (viii) SEQ ID NOs 4, 6, 8, 12, 14 and 16, respectively [FS28-256-027];
   (ix) SEQ ID NOs 53, 6, 55, 12, 14 and 34, respectively [FS28-256-001];
   (x) SEQ ID NOs 53, 6, 55, 12, 14 and 49, respectively [FS28-256-005];
   (xi) SEQ ID NOs 60, 6, 62, 12, 14 and 39, respectively [FS28-256-014];
   (xii) SEQ ID NOs 43, 6, 45, 12, 14 and 39, respectively [FS28-256-018];
   (xiii) SEQ ID NOs 67, 6, 55, 12, 14 and 39, respectively [FS28-256];
   (xiv) SEQ ID NOs 21, 23, 72, 12, 14 and 28, respectively [FS28-024-051];
   (xv) SEQ ID NOs 21, 23, 77, 12, 14 and 28, respectively [FS28-024-053]; or
   (xvi) SEQ ID NOs 21, 23, 82, 12, 14 and 28, respectively [FS28-024]; and
   wherein the CD137 antigen-binding site comprises a first sequence and a second sequence located in the AB and EF structural loops of the CH3 domain, respectively, wherein the first and second sequence have the sequence set forth in SEQ ID NOs 87 and 88, respectively.
[14] The antibody molecule or chemotherapeutic for use, method, use, or kit according to [13], wherein
   (i) the first sequence is located between positions 14 and 17 of the CH3 domain of the antibody molecule; and/or
   (ii) wherein the second sequence is located between 91 and 99 of the CH3 domain of the antibody molecule; and
   wherein the amino acid residue numbering is according to the IMGT numbering scheme.
[15] The antibody molecule or chemotherapeutic for use, method, use, or kit according to [1] to [14], wherein the antibody molecule that binds MSLN and CD137 comprises the CH3 domain sequence set forth in SEQ ID NO: 86.
[16] The antibody molecule or chemotherapeutic for use, method, use, or kit according to [15], wherein the CH3 domain comprises an additional lysine residue (K) at the immediate C-terminus of the CH3 domain sequence.
[17] The antibody molecule or chemotherapeutic for use, method, use, or kit according to [1] to [16], wherein the antibody molecule that binds MSLN and CD137 comprises the heavy chain and light chain of antibody:
   (i) FS22-172-003-AA/FS28-256-271 set forth in SEQ ID NOs 2 and 10, respectively;
   (ii) FS22-172-003-AA/FS28-024-052 set forth in SEQ ID NOs 18 and 26, respectively;
   (iii) FS22-172-003-AA/FS28-256-021 set forth in SEQ ID NOs 30 and 32, respectively;
   (iv) FS22-172-003-AA/FS28-256-012 set forth in SEQ ID NOs 36 and 37, respectively;
   (v) FS22-172-003-AA/FS28-256-023 set forth in SEQ ID NOs 41 and 32, respectively;
   (vi) FS22-172-003-AA/FS28-256-024 set forth in SEQ ID NOs 30 and 47, respectively;
   (vii) FS22-172-003-AA/FS28-256-026 set forth in SEQ ID NOs 41 and 47, respectively;
   (viii) FS22-172-003-AA/FS28-256-027 set forth in SEQ ID NOs 30 and 10, respectively;
   (ix) FS22-172-003-AA/FS28-256-001 set forth in SEQ ID NOs 51 and 32, respectively;
   (x) FS22-172-003-AA/FS28-256-005 set forth in SEQ ID NOs 51 and 47, respectively;
   (xi) FS22-172-003-AA/FS28-256-014 set forth in SEQ ID NOs 58 and 37, respectively;
   (xii) FS22-172-003-AA/FS28-256-018 set forth in SEQ ID NOs 41 and 37, respectively;
   (xiii) FS22-172-003-AA/FS28-256 set forth in SEQ ID NOs 65 and 37, respectively;
   (xiv) FS22-172-003-AA/FS28-024-051 set forth in SEQ ID NOs 70 and 26, respectively;
   (xv) FS22-172-003-AA/FS28-024-053 set forth in SEQ ID NOs 75 and 26, respectively;
   or
   (xvi) FS22-172-003-AA/FS28-024 set forth in SEQ ID NOs 80 and 26, respectively
[18] The antibody molecule or chemotherapeutic for use, method, use, or kit according to [1] to [17], wherein the antibody molecule that binds MSLN and CD137 comprises the heavy chain sequence set forth in SEQ ID NO: 2 and the light chain sequence set forth in SEQ ID NO: 10 [FS22-172-003-AA/FS28-256-271].
[19] The antibody molecule or chemotherapeutic for use, method, use, or kit according to [1] to [18], wherein the chemotherapeutic is an alkylating agent or an antimetabolite.
[20] The antibody molecule or chemotherapeutic for use, method, use, or kit according to [19], wherein the alkylating agent is selected from the group consisting of: altretamine, bendamustine, busulfan, carboplatin, carmustine, chlorambucil, cisplatin, cyclophosphamide (CPA), dacarbazine, ifosfamide, lomustine, mechlorethamine, melphalan, oxaliplatin, temozolomide, thiotepa, and trabectedin.
[21] The antibody molecule or chemotherapeutic for use, method, use, or kit according to or [20], wherein the alkylating agent is cisplatin.
[22] The antibody molecule or chemotherapeutic for use, method, use, or kit according to [18], wherein the antimetabolite is selected from the group consisting of: azacitidine, 5-fluorouracil (5-FU), 6-mercaptopurine (6-MP), capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, and a trifluridine/tipiracil combination.
[23] The antibody molecule or chemotherapeutic for use, method, use, or kit according to [20] or [22], wherein the antimetabolite is gemcitabine.
[24] The antibody molecule or chemotherapeutic for use, method, use, or kit according to [1] to [23], wherein the cancer expresses MSLN or has been determined to express MSLN.
[25] The antibody molecule or chemotherapeutic for use, method, use, or kit according to [1] to [24], wherein the cancer is selected from the group consisting of ovarian cancer, pancreatic adenocarcinoma, mesothelioma, cervical carcinoma and non-small cell lung carcinoma.
[26] The antibody molecule or chemotherapeutic for use, method, use, or kit according to [1] to [25], wherein treatment with the antibody molecule that binds MSLN and CD137 and the chemotherapeutic results in greater anti-tumor activity than monotherapy treatment with the antibody molecule that binds MSLN and CD137 or monotherapy treatment with the chemotherapeutic.
[27] The antibody molecule or chemotherapeutic for use, method, use, or kit according to [1] to [26], wherein treatment with the antibody molecule that binds MSLN and CD137 and the chemotherapeutic results in greater anti-tumor activity than the combined anti-tumor activity of monotherapy treatment with the antibody molecule that binds MSLN and CD137 and monotherapy treatment with the chemotherapeutic.
[28] The antibody molecule or chemotherapeutic for use, method, use, or kit according to [1] to [27], wherein treatment with the antibody molecule that binds MSLN and CD137 and the chemotherapeutic results in greater tumor growth retardation, reduces tumor volume reduction, increases median survival increase and/or increase in the number of complete tumor regressions than monotherapy treatment with the antibody molecule that binds MSLN and CD137 or monotherapy treatment with the chemotherapeutic.
[29] The antibody molecule or chemotherapeutic for use, method, use, or kit according to [1] to [28], wherein treatment with the antibody molecule that binds MSLN and CD137 and the chemotherapeutic results in greater tumor growth retardation, tumor volume reduction, median survival increase and/or increase in the number of complete tumor regressions than the combined tumor growth retardation, tumor volume reduction, median survival increase and/or increase in the number of complete tumor regressions of monotherapy treatment with the antibody molecule that binds MSLN and CD137 and monotherapy treatment with the chemotherapeutic.
[30] The antibody molecule or chemotherapeutic for use, method, use, or kit according to [1] to [29], wherein the antibody molecule that binds MSLN and CD137 and/or the chemotherapeutic is administered parenterally.
[31] The antibody molecule or chemotherapeutic for use, method, use, or kit according to [30], wherein the antibody molecule that binds MSLN and CD137 and/or the chemotherapeutic is administered intravenously, intramuscularly, subcutaneously, intraperitoneally or spinally.
[32] The antibody molecule or chemotherapeutic for use, method, use, or kit according to [30] or [31], wherein the antibody molecule that binds MSLN and CD137 and/or the chemotherapeutic is administered by injection or infusion.
[33] The antibody molecule or chemotherapeutic for use, method, use, or kit according to [1] to [29], wherein the antibody molecule that binds MSLN and CD137 and/or the chemotherapeutic is administered non-parenterally.
[34] The antibody molecule or chemotherapeutic for use, method, use, or kit according to [33], wherein the antibody molecule that binds MSLN and CD137 and/or the chemotherapeutic is administered orally, intranasally, vaginally, rectally, sublingually, or topically.
[35] The antibody molecule or chemotherapeutic for use, method, use, or kit according to [1] to [34], wherein the antibody molecule that binds MSLN and CD137 and the chemotherapeutic are administered to the patient concomitantly or sequentially.
[36] The antibody molecule or chemotherapeutic for use, method, use, or kit according to [35], wherein the antibody molecule that binds MSLN and CD137 and the chemotherapeutic are administered to the patient sequentially.
[37] The antibody molecule or chemotherapeutic for use, method, use, or kit according to [36], wherein the antibody molecule that binds MSLN and CD137 and the chemotherapeutic are to the patient within no more than 4 days of each other.
[38] The antibody molecule or chemotherapeutic for use, method, use, or kit according to [36], wherein the antibody molecule that binds MSLN and CD137 and the chemotherapeutic are to the patient within no more than 3 days of each other.
[39] The antibody molecule or chemotherapeutic for use, method, use, or kit according to [36], wherein the antibody molecule that binds MSLN and CD137 and the chemotherapeutic are to the patient within no more than 2 days of each other.
[40] The antibody molecule or chemotherapeutic for use, method, use, or kit according to [36], wherein the antibody molecule that binds MSLN and CD137 and the chemotherapeutic are to the patient on the same day.
[41] The antibody molecule or chemotherapeutic for use, or the method according to any one of [1] to [3] and [7] to [40], wherein the method comprises determining whether the cancer expresses MSLN and treating the individual if the cancer expresses MSLN.

### SUMMARY OF THE FIGURES

Embodiments and experiments illustrating the principles of the invention will now be discussed with reference to the accompanying figures in which:

FIG. 1 shows efficacy of treatment in a CT26 colon tumor mouse model in BALB/c mice. Progression of the tumor mouse model was measured as average tumor volume over time (A), median survival (B), % body weight change (C) and changes in individual tumor volume over time (D). Mice were treated with either anti-HEL-hIgG1-LALA, FS122m, cisplatin, or FS122m+cisplatin. Treatment with FS122m+cisplatin retarded tumor volume growth to a much greater extent than FS122m and cisplatin monotherapy when compared to the anti-HEL-hIgG1-LALA isotype control (A). FS122m+cisplatin also enhanced median survival relative to FS122m or cisplatin monotherapy (B) and induced complete tumor regression in 7 of 10 mice, compared to complete tumor regression in 2 of 10 mice treated with FS122m monotherapy and no complete tumor regression in 10 mice treated with cisplatin monotherapy (D). Changes in body weight were comparable between all treatments including the anti-HEL-hIgG1-LALA isotype control, demonstrating that all treatments were well tolerated (C). Tumor volume data were log-transformed and two-way analysis of variance (ANOVA) was performed followed by Tukey's multiple comparison test, where **=P≤0.01, ***=P≤0.001, ****=P≤0.0001. Survival is presented as median percentage survival and average tumor volume and body weight change are shown as mean±SEM.

FIG. 2 shows efficacy of treatment in a JC tumor mouse model in BALB/c mice. Progression of the tumor mouse model was measured as average tumor volume over time (A), median survival (B), % body weight change (C) and changes in individual tumor volume over time (D). Mice were treated with either anti-HEL-hIgG1-LALA, FS122m, cisplatin, or FS122m+cisplatin. Treatment with FS122m+cisplatin retarded tumor volume growth to a much greater extent than FS122m and cisplatin monotherapy when compared to the anti-HEL-hIgG1-LALA isotype control (A). FS122m+cisplatin also enhanced median survival relative to FS122m or cisplatin monotherapy (B) and induced complete tumor regression in 2 of 10 mice compared to complete tumor regression in 2 of 10 mice treated with FS122m monotherapy and no complete tumor regression in 10 mice treated with cisplatin monotherapy (D). Changes in body weight were comparable between all treatments including the anti-HEL-hIgG1-LALA isotype control, demonstrating that all treatments were well tolerated (C). Tumor volume data were log-transformed and two-way analysis of variance (ANOVA) was performed followed by Tukey's multiple comparison test, where **=P≤0.01, ***=P≤0.001, ****=P≤0.0001. Survival is presented as median percentage survival and average tumor volume and body weight change are shown as mean±SEM.

FIG. 3 shows efficacy of treatment in a JC tumor mouse model in BALB/c mice. Progression of the tumor mouse model was measured as average tumor volume over time (A), median survival (B), % body weight change (C) and changes in individual tumor volume over time (D). Mice were treated with either anti-HEL-hIgG1-LALA, FS122m, gemcitabine, or FS122m+gemcitabine. Treatment with FS122m+gemcitabine retarded tumor volume growth to a much greater extent than FS122m and gemcitabine monotherapy when compared to the anti-HEL-hIgG1-LALA isotype control (A). FS122m+gemcitabine also enhanced median survival relative to FS122m or gemcitabine monotherapy (B) and induced complete tumor regression in 5 of 10 mice compared to complete tumor regression in 1 of 10 mice treated with FS122m monotherapy and no complete tumor regression in 10 mice treated with gemcitabine monotherapy (D). Changes in body weight were comparable between all treatments including the anti-HEL-hIgG1-LALA isotype control, demonstrating that all treatments were well tolerated (C). Tumor volume data were log-transformed and two-way analysis of variance (ANOVA) was performed followed by Tukey's multiple comparison test, where **=P≤0.01, ***=P≤0.001, ****=P≤0.0001. Survival is presented as median percentage survival and average tumor volume and body weight change are shown as mean±SEM.

FIG. 4 shows efficacy of treatment in a CT26 colon tumor mouse model in BALB/c mice. Progression of the tumor mouse model was measured as average tumor volume over time (A), median survival (B), % body weight change (C) and changes in individual tumor volume over time (D). Mice were treated with either anti-HEL-hIgG1-LALA, FS122m, gemcitabine, or FS122m+gemcitabine. Treatment with FS122m+gemcitabine retarded tumor volume growth to a much greater extent than FS122m and gemcitabine monotherapy when compared to the anti-HEL-hIgG1-LALA isotype control (A). FS122m+gemcitabine also enhanced median survival relative to FS122m or gemcitabine monotherapy (B) and induced complete tumor regression in 1 of 9 mice compared to complete tumor regression in 3 of 9 mice with FS122m monotherapy and no complete tumor regression in any of 9 mice with gemcitabine monotherapy (D). Changes in body weight were comparable between all treatments including the anti-HEL-hIgG1-LALA isotype control, demonstrating that all treatments were well tolerated (C). Tumor volume data were log-transformed and two-way analysis of variance (ANOVA) was performed followed by Tukey's multiple comparison test, where **=P≤0.01, ***=P≤0.001, ****=P≤0.0001. Survival is presented as median percentage survival and average tumor volume and body weight change are shown as mean±SEM.

### DETAILED DESCRIPTION OF THE INVENTION

The present invention relates to an antibody molecule that binds MSLN and CD137 used in the treatment of cancer in a patient in combination with a chemotherapeutic. The present invention also relates to a chemotherapeutic for use in the treatment of cancer in combination with an antibody molecule that binds MSLN and CD137.

The term “bispecific” refers to a molecule that will not show any significant binding to molecules other than its two specific binding partners. The term may also refer to specific epitopes of the two binding partners, which may be carried by other antigens, in which case the antibody may also bind to the antigens carrying the specific epitopes. In a preferred embodiment, the bispecific antibody molecule does not show any significant binding activity to OX40, GITR, CD40, CEACAM-5, E-Cadherin, Thrombomodulin, or EpCAM.

The terms “antibody molecule” describe an immunoglobulin whether natural or partly or wholly synthetically produced. The antibody molecule may be human or humanised, preferably human. The antibody molecule may preferably be a monoclonal antibody. Examples of antibody molecules are the immunoglobulin isotypes, such as immunoglobulin G, and their isotypic subclasses, such as IgG1, IgG2, IgG3 and IgG4, as well as fragments thereof. The antibody molecules may be isolated, in the sense of being free from contaminants, such as antibody molecules able to bind other polypeptides and/or serum components

In the following, the term “bispecific antibody molecule” is used to refer to the antibody molecule which binds MSLN and CD137.

In one embodiment, the bispecific antibody molecule binds to MSLN and CD137 independently. In one embodiment, the bispecific antibody binds MSLN and CD137 concomitantly.

The bispecific antibody molecule may be natural or partly or wholly synthetically produced. For example, the antibody molecule may be a recombinant antibody molecule.

The bispecific antibody molecule may comprise at least one, preferably more than one, complementary determining region (CDR)-based binding site for MSLN and at least one, preferably more than one, binding site for CD137 in a constant domain of the bispecific antibody molecule, preferably at least one CH3 domain.

The bispecific antibody molecule may be an immunoglobulin or an antigen-binding fragment thereof. For example, the bispecific antibody molecule may be an IgG, IgA, IgE or IgM molecule, preferably an IgG molecule, such as an IgG1, IgG2, IgG3 or IgG4 molecule, more preferably an IgG1 or IgG2 molecule, most preferably an IgG1 molecule, or a fragment thereof. In a preferred embodiment, the bispecific antibody molecule is a complete immunoglobulin molecule.

In other embodiments, the bispecific antibody molecule may be an antigen-binding fragment comprising a CDR-based antigen-binding site for MSLN and an antigen-binding site for CD137 located in a constant domain. The antigen-binding fragment may be a scFv, Fab, Fcab, VhH, monovalent IgG, di- or triabody, IGNAR, V-NAR, hcIgG, minibody, or nanobody. For example, the antigen-binding fragment may be a scFv-Fc fusion where the scFv binds to MSLN and the Fc binds to CD137 or a minibody, which comprises an scFv joined to a CH3 domain (Hu et al. (1996), Cancer Res., 56 (13): 3055-61).

In a preferred embodiment, the bispecific antibody molecule is a mAb²™ bispecific antibody. A mAb² bispecific antibody, as referred to herein, is an IgG immunoglobulin which includes a CDR-based antigen binding site in each of its variable regions and at least one antigen binding site in a constant domain of the antibody molecule.

Antibodies and methods for their construction and use are well-known in the art and are described in, for example, Holliger and Hudson, 2005. It is possible to take monoclonal and other antibodies and use techniques of recombinant DNA technology to produce other antibodies or chimeric molecules which retain the specificity of the original antibody. Such techniques may involve introducing CDRs or variable regions of one antibody molecule into a different antibody molecule (EP-A-184187, GB 2188638A and EP-A-239400). New antibodies against known targets can be routinely produced and can arrived at without undue burden by the person skilled in the art.

A number of antibody molecule that binds MSLN and CD137 are known from WO 2020/011976. Antibody M9657 of this application is identical to antibody FS22-172-003-AA/FS28-256-271 of WO 2020/011976. Any of these antibodies can be used. The CDR-based antigen-binding site of the bispecific antibody molecule may therefore comprise the three VH CDRs or three VL CDRs, preferably the three VH CDRs and the three VL CDRs, of antibody FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, or FS22-172-003-AA/FS28-024, preferably antibody FS22-172-003-AA/FS28-256-271 or FS22-172-003-AA/FS28-024-052, most preferably antibody FS22-172-003-AA/FS28-256-271.

The sequences of the CDRs may be readily determined from the VH and VL domain sequences of an antibody molecule using routine techniques. The VH and VL domain sequences of antibodies FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, and FS22-172-003-AA/FS28-024 are described herein, and the three VH and three VL domain CDRs of said antibodies may thus be determined from said sequences. The CDR sequences may, for example, be determined according to Kabat et al., 1991 or the international ImMunoGeneTics information system (IMGT) (Lefranc et al., 2015).

The bispecific antibody molecule may carry a LALA mutation or not. The LALA mutation describes a type of mutation for disrupting the antibody effector function of an antibody molecule or fragment thereof. The LALA mutation is associated with several favourable antibody properties such as reduced toxicity (Lo et al. (2017), The Journal of Biological Chemistry, 292 (9): 3900-3908). The mutation eliminates binding of the antibody molecule or fragment thereof to Fcγ-receptors and is located in the CH2 domain. The sequences of the VH domain and VL domain, and therefore of the VH domain CDR1, CDR2 and CDR3 and the VL domain CDR1, CDR2 and CDR3, of an antibody containing the LALA mutation are the same as an antibody which does not contain the LALA mutation. The LALA mutation involves substitution of the leucine residues at positions 1.3 and 1.2 of the CH2 domain according to the IMGT numbering scheme with alanine (L1.3A and L1.2A). According to the Kabat numbering system, the LALA mutation constitutes a L247A L248A substitution. Complement activation (C1q binding) and ADCC are also known to be reduced through mutation of the proline at position 114 of the CH2 domain to alanine or glycine according to the IMGT numbering system (P114A or P114G) (Idusogie et al., 2000; Klein et al., 2016). According to the Kabat numbering system, this mutation constitutes a P348A or P348G substitution. This mutation and the LALA mutation may also be combined in order to generate antibody molecules with further reduced or no ADCC or CDC activity.

Accordingly, the bispecific antibody molecule may comprise a CH2 domain, wherein the CH2 domain comprises an alanine residue at position 1.3 and an alanine residue at position 1.2, wherein the amino acid numbering is according to the IMGT numbering system. The bispecific antibody molecule may comprise a CH2 domain, wherein the CH2 domain comprises an alanine residue at position 247 and an alanine residue at position 248, wherein the amino acid numbering is according to the Kabat numbering system. For example, the CH2 domain may have the amino acid sequence set forth in SEQ ID NO: 90. In an alternative embodiment, the antibody molecule may comprise a CH2, wherein the CH2 domain comprises an alanine residue at position 91. The antibody molecule may comprise a CH2, wherein the CH2 domain comprises an alanine residue at position 1.3, an alanine residue at position 1.2 and an alanine residue at position 114. For example, the CH2 domain may have the amino acid sequence set forth in SEQ ID NO: 92.

The VH domain CDR1, CDR2 and CDR3 sequences of the bispecific antibody molecule according to IMGT numbering may be the sequences located at positions 27-38, 56-65, and 105-117, of the VH domain of the antibody molecule, respectively.

The VH domain CDR1, CDR2 and CDR3 sequences of the bispecific antibody molecule according to Kabat numbering may be the sequences at located positions 31-35, 50-65, and 95-102 of the VH domain, respectively.

The VL domain CDR1, CDR2 and CDR3 sequences of the bispecific antibody molecule according to IMGT numbering may be the sequences located at positions 27-38, 56-65, and 105-117, of the VL domain, respectively.

The VL domain CDR1, CDR2 and CDR3 sequences of the bispecific antibody molecule according to Kabat numbering may be the sequences at located positions 24-34, 50-56, and 89-97 of the VL domain, respectively.

For example, the sequence of the VH domain CDR1, CDR2 and CDR3 of:
(i) FS22-172-003-AA/FS28-256-271 may be as set forth in SEQ ID NOs 4, 6, and 8, respectively;
(ii) FS22-172-003-AA/FS28-024-052 may be as set forth in SEQ ID NOs 20, 22, and 24, respectively;
(iii) FS22-172-003-AA/FS28-256-021 may be as set forth in SEQ ID NOs 4, 6, and 8, respectively;
(iv) FS22-172-003-AA/FS28-256-012 may be as set forth in SEQ ID NOs 4, 6, and 8, respectively;
(v) FS22-172-003-AA/FS28-256-023 may be as set forth in SEQ ID NOs 42, 6, and 44, respectively;
(vi) FS22-172-003-AA/FS28-256-024 may be as set forth in SEQ ID NOs 4, 6, and 8, respectively;
(vii) FS22-172-003-AA/FS28-256-026 may be as set forth in SEQ ID NOs 43, 6, and 45, respectively;
(viii) FS22-172-003-AA/FS28-256-027 may be as set forth in SEQ ID NOs 4, 6, and 8, respectively;
(ix) FS22-172-003-AA/FS28-256-001 may be as set forth in SEQ ID NOs 53, 6, and 55, respectively;
(x) FS22-172-003-AA/FS28-256-005 may be as set forth in SEQ ID NOs 53, 6, and 55, respectively;
(xi) FS22-172-003-AA/FS28-256-014 may be as set forth in SEQ ID NOs 60, 6, and 62, respectively;
(xii) FS22-172-003-AA/FS28-256-018 may be as set forth in SEQ ID NOs 43, 6, and 45, respectively;
(xiii) FS22-172-003-AA/FS28-256 may be as set forth in SEQ ID NOs 67, 6, and 55, respectively;
(xiv) FS22-172-003-AA/FS28-024-051 may be as set forth in SEQ ID NOs 21, 23, and 72, respectively;
(xv) FS22-172-003-AA/FS28-024-053 may be as set forth in SEQ ID NOs 21, 23, and 77, respectively; and
(xvi) FS22-172-003-AA/FS28-024 may be as set forth in SEQ ID NOs 21, 23, 82, respectively;
wherein the CDR sequences are defined according to the IMGT numbering scheme.

The sequence of the VL domain CDR1, CDR2 and CDR3 of:
(i) FS22-172-003-AA/FS28-256-271 may be as set forth in SEQ ID NOs 12, 14, and 16, respectively;
(ii) FS22-172-003-AA/FS28-024-052 may be as set forth in SEQ ID NOs 12, 14, and 18, respectively;
(iii) FS22-172-003-AA/FS28-256-021 may be as set forth in SEQ ID NOs 12, 14, and 34, respectively;
(iv) FS22-172-003-AA/FS28-256-012 may be as set forth in SEQ ID NOs 12, 14, and 39, respectively;
(v) FS22-172-003-AA/FS28-256-023 may be as set forth in SEQ ID NOs 12, 14, and 34, respectively;
(vi) FS22-172-003-AA/FS28-256-024 may be as set forth in SEQ ID NOs 12, 14, and 49, respectively;
(vii) FS22-172-003-AA/FS28-256-026 may be as set forth in SEQ ID NOs 12, 14, and 49, respectively;
(viii) FS22-172-003-AA/FS28-256-027 may be as set forth in SEQ ID NOs 12, 14, and 16, respectively;
(ix) FS22-172-003-AA/FS28-256-001 may be as set forth in SEQ ID NOs 12, 14, and 34, respectively;
(x) FS22-172-003-AA/FS28-256-005 may be as set forth in SEQ ID NOs 12, 14, and 49, respectively;
(xi) FS22-172-003-AA/FS28-256-014 may be as set forth in SEQ ID NOs 12, 14, and 39, respectively;
(xii) FS22-172-003-AA/FS28-256-018 may be as set forth in SEQ ID NOs 12, 14, and 39, respectively;
(xiii) FS22-172-003-AA/FS28-256 may be as set forth in SEQ ID NOs 12, 14, and 39, respectively;
(xiv) FS22-172-003-AA/FS28-024-051 may be as set forth in SEQ ID NOs 12, 14, and 28, respectively;
(xv) FS22-172-003-AA/FS28-024-053 may be as set forth in SEQ ID NOs 12, 14 and 28, respectively; and
(xvi) FS22-172-003-AA/FS28-024 may be as set forth in SEQ ID NOs 12, 14 and 28, respectively;
wherein the CDR sequences are defined according to the IMGT numbering scheme.

For example, the sequence of the VH domain CDR1, CDR2 and CDR3 of:
(i) FS22-172-003-AA/FS28-256-271 may be as set forth in SEQ ID NOs 5, 7, and 9, respectively;
(ii) FS22-172-003-AA/FS28-024-052 may be as set forth in SEQ ID NOs 21, 23, and 25, respectively;
(iii) FS22-172-003-AA/FS28-256-021 may be as set forth in SEQ ID NOs 5, 31 and 9, respectively;
(iv) FS22-172-003-AA/FS28-256-012 may be as set forth in SEQ ID NOs 5, 31, and 9, respectively;
(v) FS22-172-003-AA/FS28-256-023 may be as set forth in SEQ ID NOs 44, 31, and 46, respectively;
(vi) FS22-172-003-AA/FS28-256-024 may be as set forth in SEQ ID NOs 5, 31, and 9, respectively;
(vii) FS22-172-003-AA/FS28-256-026 may be as set forth in SEQ ID NOs 44, 31, and 46, respectively;
(viii) FS22-172-003-AA/FS28-256-027 may be as set forth in SEQ ID NOs 5, 31, and 9, respectively;
(ix) FS22-172-003-AA/FS28-256-001 may be as set forth in SEQ ID NOs 54, 31, and 56, respectively;
(x) FS22-172-003-AA/FS28-256-005 may be as set forth in SEQ ID NOs 54, 31, and 56, respectively;
(xi) FS22-172-003-AA/FS28-256-014 may be as set forth in SEQ ID NOs 61, 31, and 63, respectively;
(xii) FS22-172-003-AA/FS28-256-018 may be as set forth in SEQ ID NOs 44, 31, and 46, respectively;
(xiii) FS22-172-003-AA/FS28-256 may be as set forth in SEQ ID NOs 68, 31, and 56, respectively;
(xiv) FS22-172-003-AA/FS28-024-051 may be as set forth in SEQ ID NOs 22, 24, and 73, respectively;
(xv) FS22-172-003-AA/FS28-024-053 may be as set forth in SEQ ID NOs 22, 24, and 78, respectively; and
(xvi) FS22-172-003-AA/FS28-024 may be as set forth in SEQ ID NOs 22, 24, and 83, respectively;
wherein the CDR sequences are defined according to the Kabat numbering scheme.

The sequence of the VL domain CDR1, CDR2 and CDR3 of:
(i) FS22-172-003-AA/FS28-256-271 may be as set forth in SEQ ID NOs 13, 15, and 16, respectively;
(ii) FS22-172-003-AA/FS28-024-052 may be as set forth in SEQ ID NOs 13, 15, and 28, respectively;
(iii) FS22-172-003-AA/FS28-256-021 may be as set forth in SEQ ID NOs 13, 15, and 34, respectively;
(iv) FS22-172-003-AA/FS28-256-012 may be as set forth in SEQ ID NOs 13, 15, and 39, respectively;
(v) FS22-172-003-AA/FS28-256-023 may be as set forth in SEQ ID NOs 13, 15, and 34, respectively;
(vi) FS22-172-003-AA/FS28-256-024 may be as set forth in SEQ ID NOs 13, 15, and 49, respectively;
(vii) FS22-172-003-AA/FS28-256-026 may be as set forth in SEQ ID NOs 13, 15, and 49, respectively;
(viii) FS22-172-003-AA/FS28-256-027 may be as set forth in SEQ ID NOs 13, 15, and 16, respectively;
(ix) FS22-172-003-AA/FS28-256-001 may be as set forth in SEQ ID NOs 13, 15, and 34, respectively;
(x) FS22-172-003-AA/FS28-256-005 may be as set forth in SEQ ID NOs 13, 15, and 49, respectively;
(xi) FS22-172-003-AA/FS28-256-014 may be as set forth in SEQ ID NOs 13, 15, and 39, respectively;
(xii) FS22-172-003-AA/FS28-256-018 may be as set forth in SEQ ID NOs 13, 15, and 39, respectively;
(xiii) FS22-172-003-AA/FS28-256 may be as set forth in SEQ ID NOs 13, 15, and 39, respectively;
(xiv) FS22-172-003-AA/FS28-024-051 may be as set forth in SEQ ID NOs 13, 15, and 28, respectively;
(xv) FS22-172-003-AA/FS28-024-053 may be as set forth in SEQ ID NOs 13, 15, and 28, respectively; and
(xvi) FS22-172-003-AA/FS28-024 may be as set forth in SEQ ID NOs 13, 15, and 28, respectively;
wherein the CDR sequences are defined according to the Kabat numbering scheme.

The CDR-based antigen-binding site may comprise the VH or VL domains, preferably the VH and VL domains, of antibody of antibody FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, or FS22-172-003-AA/FS28-024, preferably antibody FS22-172-003-AA/FS28-256-271 or FS22-172-003-AA/FS28-024-052, most preferably antibody FS22-172-003-AA/FS28-256-271.

The VH domain of antibodies FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, and FS22-172-003-AA/FS28-024 may have the sequence set forth in SEQ ID NOs 3, 19, 3, 3, 42, 3, 42, 3, 52, 52, 59, 42, 66, 71, 76, and 81, respectively.

The VL domain of antibodies FS22-172-003-AA/FS28-256-271, FS22-172-003-AA/FS28-024-052, FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, and FS22-172-003-AA/FS28-024 may have the sequence set forth in SEQ ID NOs 11, 27, 33, 38, 33, 48, 48, 11, 33, 48, 38, 38, 38, 27, 27, and 27, respectively.

In one embodiment, the bispecific antibody molecule of the invention comprises a CD137 antigen-binding site. The CD137 antigen-binding site may be located in a constant domain of the antibody molecule, preferably a CH3 domain. The CD137 antigen-binding may comprises one or more modified structural loops in a constant domain of the antibody molecule. Engineering antibody constant domain structural loops to create antigen-binding sites for target antigens is known in the art and is described, for example, Wozniak-Knopp G et al. (2010) Protein Eng Des. 23 (4): 289-297; WO2006/072620 and WO2009/132876. The CD137 constant domain antigen-binding site comprised in the antibody molecules of the invention was identified following an extensive selection and affinity maturation program, and preferentially binds to dimeric rather than monomeric human CD137.

The CD137 antigen-binding site of the bispecific antibody molecule may comprise a first and second sequence, wherein the first and second sequences are located in the AB and EF structural loops of the constant domain, preferably the CH3 domain, of the bispecific antibody molecule, respectively. The first sequence and second sequence are preferably the first and second sequence of FS22-172-003 set forth in SEQ ID NOs 87 and 88, respectively. The first and second sequences are preferably located between positions 14 and 17, and positions 91 and 99, of the CH3 domain of the bispecific antibody molecule, respectively, wherein the residue numbering is according to IMGT numbering. The CD loop sequence of the bispecific antibody molecule is preferably unmodified, i.e. wild-type.

The CD loop sequence therefore preferably has the sequence set forth in SEQ ID NO: 89. The CD loop sequence is preferably located at positions 43 to 78 of the CH3 domain of the bispecific antibody molecule, wherein the residue numbering is according to IMGT numbering.

In a preferred embodiment, the bispecific antibody molecule comprises a CH3 domain which comprises, has, or consists of the CH3 domain sequence of FS22-172-003 set forth in SEQ ID NO: 86. The CH3 domain of the bispecific antibody molecule may optionally comprise an additional lysine residue (K) at the immediate C-terminus of the CH3 domain sequence.

In a preferred embodiment, the antibody molecule comprises the heavy chain and/or light chain, preferably the heavy chain and light chain, of antibody:
(i) FS22-172-003-AA/FS28-256-271 set forth in SEQ ID NOs 2 and 10, respectively;
(ii) FS22-172-003-AA/FS28-024-052 set forth in SEQ ID NOs 18 and 26, respectively;
(iii) FS22-172-003-AA/FS28-256-021 set forth in SEQ ID NOs 30 and 32, respectively;
(iv) FS22-172-003-AA/FS28-256-012 set forth in SEQ ID NOs 36 and 37, respectively;
(v) FS22-172-003-AA/FS28-256-023 set forth in SEQ ID NOs 41 and 32, respectively;
(vi) FS22-172-003-AA/FS28-256-024 set forth in SEQ ID NOs 30 and 47, respectively;
(vii) FS22-172-003-AA/FS28-256-026 set forth in SEQ ID NOs 41 and 47, respectively;
(viii) FS22-172-003-AA/FS28-256-027 set forth in SEQ ID NOs 30 and 10, respectively;
(ix) FS22-172-003-AA/FS28-256-001 set forth in SEQ ID NOs 51 and 32, respectively;
(x) FS22-172-003-AA/FS28-256-005 set forth in SEQ ID NOs 51 and 47, respectively;
(xi) FS22-172-003-AA/FS28-256-014 set forth in SEQ ID NOs 58 and 37, respectively;
(xii) FS22-172-003-AA/FS28-256-018 set forth in SEQ ID NOs 41 and 37, respectively;
(xiii) FS22-172-003-AA/FS28-256 set forth in SEQ ID NOs 65 and 37, respectively;
(xiv) FS22-172-003-AA/FS28-024-051 set forth in SEQ ID NOs 70 and 26, respectively;
(xv) FS22-172-003-AA/FS28-024-053 set forth in SEQ ID NOs 75 and 26, respectively;
or
(xvi) FS22-172-003-AA/FS28-024 set forth in SEQ ID NOs 80 and 26, respectively.

In a more preferred embodiment, the bispecific antibody molecule comprises the heavy chain and/or light chain, preferably the heavy chain and light chain, of: antibody FS22-172-003-AA/FS28-256-271 or FS22-172-003-AA/FS28-024-052, most preferably antibody FS22-172-003-AA/FS28-256-271, wherein the heavy and light chain sequences of these antibodies are as set out above.

The bispecific antibody molecules of the present invention may also comprise variants of a first, second or third sequence, AB, CD or EF structural loop sequence, CH3 domain, CH2 domain, CDR, VH domain, VL domain, light chain and/or heavy chain sequences disclosed herein. Suitable variants can be obtained by means of methods of sequence alteration, or mutation, and screening. In a preferred embodiment, an antibody molecule comprising one or more variant sequences retains one or more of the functional characteristics of the parent antibody molecule, such as binding specificity and/or binding affinity for MSLN and CD137. For example, an antibody molecule comprising one or more variant sequences preferably binds to MSLN and/or CD137 with the same affinity, or a higher affinity, than the (parent) antibody molecule. The parent antibody molecule is an antibody molecule which does not comprise the amino acid substitution(s), deletion(s), and/or insertion(s) which have been incorporated into the variant antibody molecule.

An antibody molecule which comprises CDRs 1-6, the VH domain, and/or the heavy chain of antibody FS22-172-003-AA/FS28-256-271, FS22-172-003-AA/FS28-024-052 FS22-172-003-AA/FS28-256-021, FS22-172-003-AA/FS28-256-012, FS22-172-003-AA/FS28-256-023, FS22-172-003-AA/FS28-256-024, FS22-172-003-AA/FS28-256-026, FS22-172-003-AA/FS28-256-027, FS22-172-003-AA/FS28-256-001, FS22-172-003-AA/FS28-256-005, FS22-172-003-AA/FS28-256-014, FS22-172-003-AA/FS28-256-018, FS22-172-003-AA/FS28-256, FS22-172-003-AA/FS28-024-051, FS22-172-003-AA/FS28-024-053, or FS22-172-003-AA/FS28-024 may comprise an amino acid substitution at position 55 or 57 of the VH domain, wherein the amino acid residue numbering is according to the IMGT numbering scheme.

For example, the antibody molecule may comprise CDRs 1-6, the VH domain, and/or the heavy chain of antibody FS22-172-003-AA/FS28-256-027, wherein the antibody molecule comprises an amino acid substitution at position 55 of the VH domain, and wherein the amino acid residue numbering is according to the IMGT numbering scheme. For example, an antibody molecule of the invention may comprise a first, second or third sequence, AB, CD or EF structural loop sequence, CH3 domain, CH2 domain, CDR, VH domain, VL domain, light chain and/or heavy chain sequence which has at least 70%, at least 75%, at least 80%, at least 85%, at least 90%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a structural loop, CH3 domain, CH2 domain, CDR, VH domain, VL domain, light chain or heavy chain sequence disclosed herein.

In a preferred embodiment, the bispecific antibody molecule of the invention comprises a CH3 domain sequence which has at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a CH3 domain as disclosed herein.

In a further preferred embodiment, the bispecific antibody molecule has or comprises a CH2 domain sequence, which has at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity to a CH2 domain as disclosed herein.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equalling 12 and a gap extension penalty equalling 4. Use of GAP may be preferred but other algorithms may be used, e.g. BLAST (which uses the method of Altschul et al., 1990), FASTA (which uses the method of Pearson and Lipman, 1988), or the Smith-Waterman algorithm (Smith and Waterman, 1981), or the TBLASTN program, of Altschul et al., 1990 supra, generally employing default parameters. In particular, the psi-Blast algorithm (Altschul et al., 1997) may be used.

The bispecific antibody molecule of the invention may also comprise a first, second or third sequence, AB, CD or EF structural loop sequence, CH3 domain, CH2 domain, VH domain, VL domain, light chain and/or heavy chain which has one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with a first, second or third sequence, AB, CD or EF structural loop sequence, CH3 domain, CH2 domain, Fcab, CDR, VH domain, VL domain, light chain or heavy chain sequence disclosed herein. In particular, alterations may be made in one or more framework regions of the antibody molecule outside the VH and VL domain sequences and/or in one or more framework regions of the CH3 domain. For example, the alterations may be in the CH3 domain outside of the sequences described herein as a first, second and third sequences, or as AB, CD or EF structural loop sequences.

The bispecific antibody molecule may comprise a VH CDR1, VH CDR2, VH CDR3, VL CDR1, VL CDR2, and/or VL CDR3 which has one or more amino acid sequence alterations (addition, deletion, substitution and/or insertion of an amino acid residue), preferably 3 alterations or fewer, 2 alterations or fewer, or 1 alteration compared with the VH CDR1, VH CDR2, VH CDR3, VL CDR1, VL CDR2, and/or VL CDR3 as disclosed herein.

In a preferred embodiment, the bispecific antibody molecule of the invention comprises a CH3 domain sequence with one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the CH3 domain as disclosed herein.

In preferred embodiments in which one or more amino acids are substituted with another amino acid, the substitutions are conservative substitutions, for example according to the following Table. In some embodiments, amino acids in the same category in the middle column are substituted for one another, i.e. a non-polar amino acid is substituted with another non-polar amino acid, for example. In some embodiments, amino acids in the same line in the rightmost column are substituted for one another.

| | | |
|---|---|---|
| ALIPHATIC | Non-polar | G A P |
| | | I L V |
| | Polar - uncharged | C S T M |
| | | N Q |
| | Polar - charged | D E |
| | | K R |
| AROMATIC | | H F W Y |

In some embodiments, substitution(s) are functionally conservative. That is, in some embodiments the substitution does not affect (or does not substantially affect) one or more functional properties (e.g. binding affinity) of the antibody molecule comprising the substitution as compared to the equivalent unsubstituted antibody molecule.

The term “chemotherapeutic” describes a broad range of agents used in the treatment of cancer. Chemotherapeutic agents may be naturally occurring compounds or may be partially or wholly synthetically produced.

For example, the chemotherapeutic may be an alkylating agent, a nitrosourea, an antimetabolite, a cytotoxic antibiotic, a topoisomerase inhibitor, a mitotic inhibitor, a corticosteroid or any of a group of agents including trans-retinoic acid, arsenic trioxide, asparaginase, eribulin, hydroxyurea, ixabepilone, mitotane, omacetaxine, pegaspargase, procarbazine, romidepsin, or vorinostat. The topoisomerase inhibitor may be a type I topoisomerase inhibitor or a type II topoisomerase inhibitor. The mitotic inhibitor may be a taxane or a vinca alkaloid.

Preferably, the chemotherapeutic is an alkylating agent or an antimetabolite. Preferably, the antimetabolite is selected from the group including azacitidine, 5-fluorouracil (5-FU), 6-mercaptopurine (6-MP), capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, and a trifluridine/tipiracil combination. More preferably, the antimetabolite is gemcitabine. Preferably, the alkylating agent is selected from the group including altretamine, bendamustine, busulfan, carboplatin, carmustine, chlorambucil, cisplatin, cyclophosphamide (CPA), dacarbazine, ifosfamide, lomustine, mechlorethamine, melphalan, oxaliplatin, temozolomide, thiotepa, and trabectedin. More preferably, the alkylating agent is cisplatin.

Preferably, the nitrosourea is selected from the group including camustine, lomustine and streptozocin.

Preferably, the cytotoxic antibiotic is selected from the group consisting of daunorubicin, doxorubicin, liposomal doxorubicin, epirubicin, idarubicin, valrubicin, bleomycin, dactinomycin, and mitomycin C. Preferably, the type I topoisomerase inhibitor is selected from the group including irinotecan, liposomal irinotecan, and topotecan. Preferably, the type II topoisomerase inhibitor is selected from the group including etoposide, mitoxantrone, and teniposide. Preferably, the taxane is selected from the group including cabazitaxel, docetaxel, nab-paclitaxel, and paclitaxel. Preferably, the vinca alkaloid is selected from the group including vinblastine, vincristine, liposomal vincristine, and vinorelbine. Preferably, the corticosteroid is selected from the group including prednisone, methylprednisolone, and dexamethasone.

The word “tumor” refers to a mass of cells of abnormal size and/or composition resulting from increased proliferation and/or prolonged survival of cells. Tumors may be benign or malign. In the latter case they are referred to as “cancer”. A “tumor” cell therefore is a cell which possesses an abnormally increased ability to divide and/or to resist cell death compared to other cells of the same cell type.

Cancer is characterised by the abnormal proliferation of malignant tumor cells. Where a particular type of cancer, such as ovarian cancer, is referred to, this refers to an abnormal proliferation of malignant cells of the relevant tissue, such as breast tissue. A secondary cancer which is located in the breast but is the result of abnormal proliferation of malignant cells of another tissue, such as ovarian tissue, is not a breast cancer as referred to herein but an ovarian cancer.

MSLN is expressed on the surface of some tumor cells and high expression levels of soluble MSLN have been correlated with poor prognosis in several cancers. Anti-MSLN antibodies have been investigated as anti-cancer therapeutics. These anti-MSLN antibodies either induce direct cell killing through their ADCC activity or are used in the form of ADCs.

Accordingly, the cancer to be treated using a bispecific antibody molecule that binds MSLN and CD137 in combination with a chemotherapeutic therefore preferably expresses, or has been determined to express, MSLN. More preferably, cells of the cancer to be treated comprise, or have been determined to comprise, MSLN at their cell surface, i.e. to comprise cell-surface bound MSLN.

The cancer preferably comprises, or has been determined to comprise, tumor infiltrating lymphocytes (TILs) that express CD137. Specifically, the TILs preferably comprise, or have been determined to comprise, CD137 on their cell surface.

The cancer may be a primary or a secondary cancer. Thus, an antibody molecule that binds MSLN and CD137 as described herein may be for use in a method of treating cancer in an individual in combination with a chemotherapeutic, wherein the cancer is a primary tumour and/or a tumor metastasis.

The cancer to be treated may be a solid cancer.

As mentioned above, the cancer to be treated may be a cancer that expresses MSLN or has been determined to express MSLN. Preferably, the cancer is selected from the group including ovarian cancer, pancreatic adenocarcinoma, mesothelioma, cervical carcinoma and non-small cell lung carcinomas.

The patient to be treated may be selected for treatment if the cancer expresses MSLN. The patient may be selected if the cancer has been determined to express MSLN. Preferably, the patient is selected for treatment if the cancer is any one of ovarian cancer, pancreatic adenocarcinoma, mesothelioma, cervical carcinoma or non-small cell lung carcinomas and expressed MSLN.

The present inventors showed that combined treatment with FS122m (SEQ ID NO: 84 and SEQ ID NO: 85) and either cisplatin or gemcitabine resulted in greater tumor growth retardation, tumor volume reduction, increased median survival and an increase in the percentage of mice with complete tumor regression in ST26 and JC mouse tumor models than the combined tumor growth retardation, tumor volume reduction, increase in median survival and increase in the percentage of mice with complete tumor regression observed when mice were treated with either FS122m or with cisplatin or gemcitabine alone. Accordingly, the present inventors showed that combined treatment with FS122m and either cisplatin or gemcitabine retarded tumor growth, reduced tumor volume, increased median survival and increased the percentage of mice with complete tumor regression in ST26 and JC mouse tumor models in a synergistic manner.

In one embodiment, the antibody molecule that binds MSLN and CD137 therefore in combination with the chemotherapeutic decreases tumor volume, delays tumor growth, increases the survival rate, and/or increases the percentage of patients with complete tumor regression. Preferably, the antibody molecule that binds MSLN and CD137 in combination with the chemotherapeutic decreases tumor volume, delays tumor growth, increases survival rate, and/or increased the percentage of patients with complete tumor regression to a statistically significantly greater extent than monotherapy with the bispecific antibody molecule that binds MSLN and CD137 or monotherapy with the chemotherapeutic. More preferably, the bispecific antibody molecule that binds MSLN and CD137 in combination with the chemotherapeutic decreases tumor volume, delays tumor growth, increases survival rate and/or increases the percentage of patients with complete tumor regression in a synergistic manner, i.e. compared with the combined tumor growth retardation, tumor volume reduction, median survival increase and/or increase in the percentage of patients with complete tumor regression when patients are treated with the antibody molecule that binds MSLN and CD137 or the chemotherapeutic alone.

In one embodiment, the antibody molecule that binds MSLN and CD137 and the chemotherapeutic are administered as a first (“front”) line of treatment (e.g., the initial or first treatment). In another embodiment, the antibody molecule that binds MSLN and CD137 and the chemotherapeutic are administered as a second line of treatment (e.g., after initial treatment with the same or a different therapeutic, including after relapse and/or where the first treatment has failed).

“Administering” as used herein refers to the physical introduction of a therapeutic agent to a subject, using any of the various methods and delivery systems known to those skilled in the art. Exemplary routes of administration for the antibody molecule that binds MSLN and CD137 and/or the chemotherapeutic include intravenous, intramuscular, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion, as well as in vivo electroporation. A therapeutic agent may be administered via a non-parenteral route. Non-parenteral routes include a topical, epidermal or mucosal route of administration, for example, orally, intranasally, vaginally, rectally, sublingually, or topically.

Accordingly, in some embodiments the antibody molecule that binds MSLN and CD137 and/or the chemotherapeutic are administered parenterally. The antibody molecule that binds MSLN and CD137 and/or the chemotherapeutic may be administered intravenously, intramuscularly, subcutaneously, intraperitoneally or spinally. Alternatively, the antibody molecule that binds MSLN and CD137 and/or the chemotherapeutic may be administered by injection or infusion.

In other embodiments, the embodiments the antibody molecule that binds MSLN and CD137 and/or the chemotherapeutic are administered non-parenterally. The antibody molecule that binds MSLN and CD137 and/or the chemotherapeutic may be administered orally, intranasally, vaginally, rectally, sublingually, or topically.

“Concomitant administration” describes the simultaneous administration of the antibody molecule that binds MSLN and CD137 and the chemotherapeutic in the same or in separate formulations. “Sequential administration” refers to the timely separated administration of the bispecific antibody molecule and the chemotherapeutic in separate formulations.

Thus, the antibody molecule that binds MSLN and CD137 and the chemotherapeutic may be part of the same formulation or part of separate formulations. Preferably, the antibody molecule that binds MSLN and CD137 and the chemotherapeutic are provided as separate formulations.

In one embodiment of the invention, the antibody molecule that binds MSLN and CD137 and the chemotherapeutic are administered concomitantly. For example, the antibody molecule that binds MSLN and CD137 may be administered with the chemotherapeutic in the same formulation. Alternatively, the antibody molecule that binds MSLN and CD137 and the chemotherapeutic may be administered in separate formulations immediately before or after one another.

Preferably, the antibody molecule that binds MSLN and CD137 and the chemotherapeutic are administered to the patient sequentially. More preferably, the antibody molecule that binds MSLN and CD137 and the chemotherapeutic are administered to the patient within 4 days of each other, preferably within 3 days of each other, more preferably within 2 days of each other, or sequentially on the same day.

The present invention also relates to a method of treating cancer comprising administering to the individual in need thereof an antibody molecule that binds MSLN and CD137 and a chemotherapeutic.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of antibody molecule, the method of administration, the scheduling of administration and other factors known to medical practitioners. Prescription of treatment, e.g. decisions on dosage etc., is within the responsibility of general practitioners and other medical doctors, and may depend on the severity of the symptoms and/or progression of a disease being treated. Appropriate doses of antibody molecules are well known in the art (Ledermann et al., 1991; Bagshawe et al., 1991). A therapeutically effective amount or suitable dose of an antibody molecule can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the antibody molecule.

Accordingly, the invention may relate to a method of treating cancer comprising administering to the individual in need thereof a therapeutically effective amount of an antibody molecule that binds MSLN and CD137 and a therapeutically effective amount of a chemotherapeutic.

Also provided is a kit comprising an antibody molecule that binds MSLN and CD137 and a chemotherapeutic. Preferably, the kit comprises a antibody molecule that binds MSLN and CD137 and a pharmaceutically acceptable excipient and a chemotherapeutic and a pharmaceutically acceptable excipient.

The kit may be a package comprising a first container and a second container, the first container comprising the antibody molecule that binds MSLN and CD137, the second container comprising the chemotherapeutic. The package may comprise instructions for use of the antibody molecule that binds MSLN and CD137 in combination with the chemotherapeutic for the treatment of cancer in an individual.

In one embodiment, the kit may be a package comprising at least one dose of a medicament comprising the antibody molecule that binds MSLN and CD137 and one dose of a medicament comprising the chemotherapeutic. Preferably, the kit comprises at least one dose of a medicament comprising the antibody molecule that binds MSLN and CD137 and a pharmaceutically acceptable excipient and one dose of a medicament comprising the chemotherapeutic and a pharmaceutically acceptable excipient. More preferably, the kit comprises a package insert comprising instructions for treating cancer in an individual using the medicaments.

In another embodiment, the kit may be a package comprising a first container and a second container, the first container comprising the antibody molecule that binds MSLN and CD137, the second container comprising the chemotherapeutic. The first container may comprise at least one dose of a medicament comprising the antibody molecule that binds MSLN and CD137 and a pharmaceutically acceptable excipient and the second container may compromise at least one dose of a medicament comprising the chemotherapeutic. The package may further comprise an insert comprising instructions for using the medicaments for the treatment of cancer in an individual.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### EXAMPLES

### Materials and Methods

The efficacy of the combination of an antibody molecule that binds MSLN and CD137 and a chemotherapeutic in enhancing an anti-cancer immune response compared to monotherapy with either the antibody molecule that binds MSLN and CD137 or the chemotherapeutic was assessed by studying the average tumour volume over time and prolonged survival in mouse tumour models. These mouse tumor models are described in more detail below.

CT26 cells were obtained from ATCC and cultured in RPMI media containing 2 mM L-glutamine, 10 mM HEPES, 1 mM sodium pyruvate, 4500 mg/L glucose, 1500 mg/L sodium bicarbonate, and 10% fetal bovine serum (FBS). JC cells were obtained from ATCC and were maintained in vitro as a monolayer culture at WuXi in RPMI-1640 supplemented with 10% heat-inactivated FBS. These tumor cell lines were individually maintained in vitro as monolayer cultures and grown at 37° C. in an atmosphere of 5% CO₂. The cells were routinely sub-cultured twice weekly by TrypLE treatment. The cells growing in an exponential growth phase were harvested and counted for tumor inoculation. Wild-type BALB/c female mice were purchased from Charles River Laboratories, or from Lingchang Biological Technology Co. LTD. All mice were 8-12 weeks old at the start of studies and were housed and maintained as described below, under conditions that conform to The Guide for the Care and Use of Laboratory Animals, 8th Edition. All animal experiments were performed in accordance with EMD Serono Research Institutional (protocol 17-008, 20-005) and Wuxi AppTec Animal Care and Use Committee (IACUC) guidelines.

Upon arrival at the research institute's vivarium, all animals received a detailed physical examination, including body weight measurement, by the research staff. All animals were found to be in satisfactory health. Animals were housed in a specific pathogen free barrier animal facility at EMD Serono. The mice were kept in individual ventilation cages at constant temperature and humidity with 5 animals in each cage. The identification labels for each cage contained the following information: number of animals, sex, strain, date received, treatment, study number, group number and the starting date of the treatment. Animals were marked by ear notches or ear tags. Animal holding rooms were maintained at 20-26° C. and 40-70% humidity. Lights were on a 12-hour light/dark cycle. Animals had free access to a standard certified commercial laboratory diet. Maximum allowable concentrations of contaminants in the diet are controlled and routinely analyzed by the manufacturers. Autoclaved municipal tap water, suitable for human consumption will be available to the animals ad libitum. It is considered that there are no known contaminants in the dietary materials that could influence the tumor growth. A period of approximately one week was allowed between animal arrival and tumor inoculation in order to allow the animals to acclimatize to the laboratory environment. All the procedures related to animal handling, care, and treatment in the study were performed according to the guidelines approved by the institutional animal care and use committees (IACUCs) of EMD Serono and following the guidance of the Association for Assessment and Accreditation of Laboratory Animal Care (AAALAC).

CT26 and JC tumor models were used to investigate the antitumor effect of a combination of an antibody molecule that binds MSLN and CD137 and a chemotherapeutic compared to monotherapy with the antibody molecule that binds MSLN and CD137 or the chemotherapeutic. Mice were treated with the anti-mMSLN-mCD137-huIgG1-LALA antibody molecule FS122m in combination with either of the two chemotherapeutics cisplatin or gemcitabine. For the CT26 model, female BALB/c mice were inoculated via subcutaneous (sc) injection in the right flank with 0.3×10⁶ CT26 cells in 0.1 mL of PBS. Mice were randomized to treatment groups (n=10 mice/group) when the average tumor volume reached approximately 50-100 mm³. For the JC model, female BALB/c mice were inoculated via sc injection at the right upper flank with JC tumor cells (5×10⁶) in 0.1 mL of PBS. Treatments were started on day 14 after tumor inoculation when the average tumor size reached approximately 80 mm³. FS122m and anti-HEL hIgG1 LALA isotype control were given as 5 mg/kg on the first, third and fifth day of treatment (Days 0, 2, and 4) via intraperitoneal (ip) injection. Cisplatin was given on Day 0 at 10 mg/kg via ip injection, and gemcitabine was given on Day 0 at 120 mg/kg via ip injection.

Mortality checks were performed once daily. Animals were checked daily for any effects of tumor growth and treatments on normal behavior such as mobility, food and water consumption (by looking only), eye/hair matting and any other abnormal effect as stated in the protocol. Body weights were recorded twice weekly and any mouse with a 20% loss of initial body weight loss was humanely sacrificed. Mice were humanely euthanized if their subcutaneous tumors reached a volume of 2500 mm³.

If tumor ulceration occurred, animals with ulcerated tumors were monitored at least 3 times per week with increasing frequency, up to once daily, depending upon clinical signs. Ulcerated tumors that did not scab over were cleaned with an appropriate wound cleansing solution (e.g., Novalsan). Antibiotic cream was applied to the ulceration/lesion only if directed by the veterinary staff.

Criteria for euthanasia included the lesion not healing or forming a scab within 1 week, the lesion being greater than 5 mm diameter, the lesion becoming cavitated or developing signs of infection (such as presence of pus) or bleeding, or the animal showing signs of discomfort (e.g. excessive licking and biting directed at the site) or systemic signs of illness (lethargy, decreased activity, decreased food consumption, decreased body condition or weight loss). The veterinary staff was contacted to discuss any possible exceptions.

Animals were euthanized if they were found to be moribund. Clinical examples of morbidity may include the animal being hunched, persistent recumbency and lack of response to handling or other stimuli, signs of severe organ or system failure, emaciation, hypothermia, CNS deficits (convulsions), respiratory symptoms (rapid respiratory rate, labored breathing, coughing, rales), gastrointestinal symptoms (diarrhea lasting >2 days, jaundice). Any animal that exhibited the above clinical issues was humanely sacrificed by CO₂.

Body weight was measured and recorded twice weekly until an endpoint was reached. Tumor sizes were measured twice per week in three dimensions using a caliper, and the volume was expressed in mm³ using the formula: width×length×height×0.5236.

Differences in tumor growth between treatment groups were determined using two-way analysis of variance (ANOVA) and Tukey's Multiple Comparisons Test. The significance of survival was determined using Log-rank (Mantel-Cox) test. All analyses were conducted using the GraphPad Prism software package (Prism 5 for Windows, Version 8.0, GraphPad Software Inc., San Diego, CA) and statistical significance was accepted at the p≤0.05 level.

### Example 1: In Vivo Proof of Concept

To test if the combination of an antibody molecule that binds MSLN and CD137 and a chemotherapeutic showed improved anti-tumor activity over monotherapy with the bispecific antibody molecule and monotherapy with the chemotherapeutic, in vivo mouse tumor model were treated with the antibody molecule that binds MSLN and CD137 in combination with the chemotherapeutic. The outcome on tumor growth retardation, tumor volume reduction and survival rate was then compared to monotherapy with the antibody molecule that binds MSLN and CD137 and monotherapy with the chemotherapeutic.

Due to the lack of cross reactivity between the bispecific anti-MSLN/CD137 antibody molecule M9657 and mouse MSLN and mouse CD137, anti-mMSLN-mCD137-huIgG1-LALA (FS112m) bispecific antibody having a similar binding affinity for mouse CD137 as that of M9657 for human CD137 was developed as a surrogate antibody (SEQ ID NO: 84 and 85). Cisplatin and gemcitabine are two chemotherapeutics belonging to the group of alkylating agents and antimetabolites, respectively. FS112m was combined with either cisplatin or gemcitabine and the tumor efficacy of the combination compared to monotherapy with FS122m and to monotherapy with cisplatin or gemcitabine in two different mouse tumor models. To this end, female mice were inoculated with either CT26 colon cancer or JC breast cancer cells and randomized to treatment groups when the average tumor volume reached approximately 50-100 mm³. Mice were inoculated, treated and treatment terminated according to the method described in Materials and Methods.

### 1.1 FS221m and Cisplatin Combination

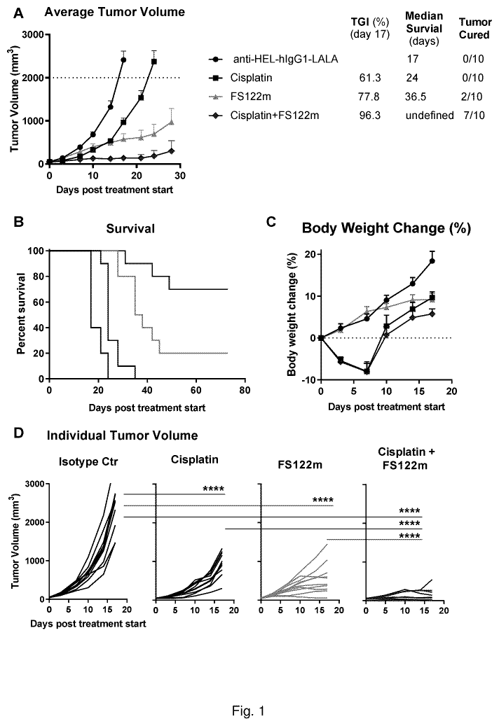

The antitumor efficacy of FS122m in combination with cisplatin was first evaluated in the CT26 colon tumor model in BALB/c mice. FS122m and cisplatin monotherapies induced significant tumor growth inhibition (TGI) (61.3% and 77.8%, respectively) relative to the isotype control (P≤0.0001 for both, Day 17) and prolonged median survival (24 and 36.5 days, respectively) relative to the isotype control (17 days) (FIGS. 1A, B, and D). Combination of FS122m and cisplatin resulted in greater TGI (96.3%) than the TGI observed with FS122m monotherapy (P≤0.0001, Day 17) and cisplatin monotherapy (P≤0.0001, Day 17) (FIGS. 1A and D). The combination of FS122m and cisplatin also prolonged median survival (FIG. 1B) and induced complete tumor regression in 7 of 10 mice, compared to complete tumor regression in 2 of 10 mice treated with FS122m monotherapy and no complete tumor regression in 10 mice treated with cisplatin monotherapy (FIG. 1D).

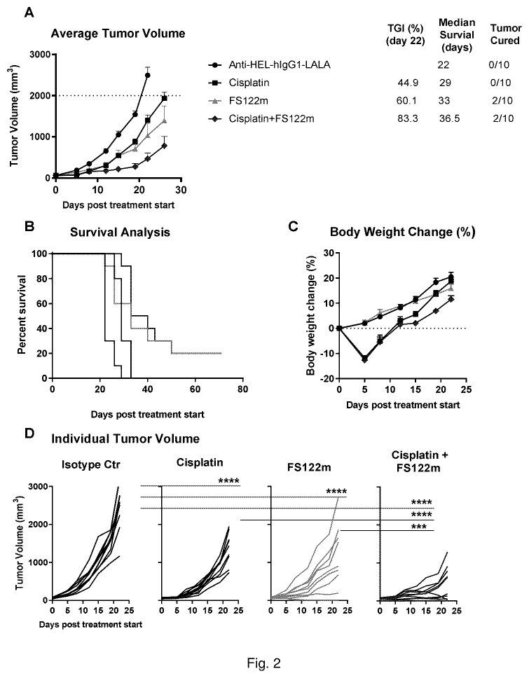

In JC tumor-bearing mice, treatment with cisplatin induced marginal, but statistically significant TGI (44.9%, P≤0.0001) on Day 22 post treatment initiation compared with the isotype control, and FS122m monotherapy induced moderate TGI (60.1% P≤0.0001) on Day 22 (FIGS. 2A and D). Cisplatin and FS122m monotherapies prolonged survival relative to the isotype control (22 days vs. 29 and 33 days, respectively) (FIG. 2B). The combination of FS122m and cisplatin resulted in greater TGI (83.3%) than the TGI observed with cisplatin monotherapy (P≤0.0001) and FS122m monotherapy (P≤0.001) (FIGS. 2A and D). The combination of FS122m and cisplatin also prolonged median survival (36.5 days) (FIG. 2B) and induced complete tumor regression in 2 of 10 mice compared to complete tumor regression in 2 of 10 mice treated with FS122m monotherapy and no complete tumor regression in 10 mice treated with cisplatin monotherapy (FIG. 2D).

In both CT26 colon tumor- and JC tumor-bearing mice, the combination of FS122m and cisplatin resulted in greater TGI than the TGI observed when mice were treated with either FS122m monotherapy or cisplatin monotherapy. The difference compared with FS122m and cisplatin monotherapy was particularly stark in the CT26 mouse tumor model, where the combination of FS122m and cisplatin resulted in complete tumor regression in 7 out of 10 mice compared with 2 out of 10 mice with FS122m monotherapy and no mice in the cisplatin monotherapy group (FIG. 1D). No significant body weight loss was observed in either mouse tumor model after treatment with the combination of FS122m and cisplatin, suggesting that the combination treatment was well tolerated in the animals (FIGS. 1C and 2C).

### 1.2 FS221m and Gemcitabine Combination

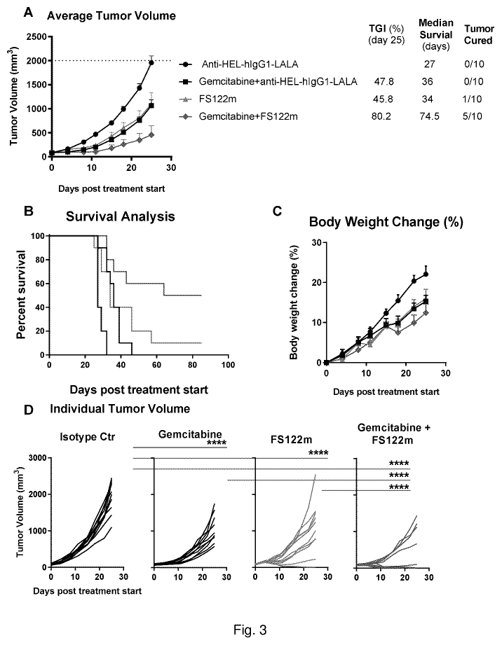

The antitumor efficacy of FS122m in combination with gemcitabine was first evaluated in JC tumor bearing mice. Treatment with gemcitabine or FS122m monotherapy induced marginal, but statistically significant TGI (47.8% and 45.8%, respectively) relative to the isotype control (P≤0.0001 for both, Day 25) (FIGS. 3A and D). Gemcitabine and FS122m monotherapies prolonged survival relative to the isotype control (27 days vs. 36 and 34 days, respectively) (FIG. 3B). The combination of FS122m and gemcitabine resulted in greater TGI (80.2%) than gemcitabine monotherapy (P≤0.0001) and FS122m monotherapy (P≤0.001) (FIG. 3A). Combination of FS122m and gemcitabine also prolonged median survival (74.5 days) (FIG. 3B) and induced complete tumor regression in 5 of 10 mice compared to complete tumor regression in 1 of 10 mice treated with FS122m monotherapy and no complete tumor regression in 10 mice treated with cisplatin monotherapy (FIG. 3D).

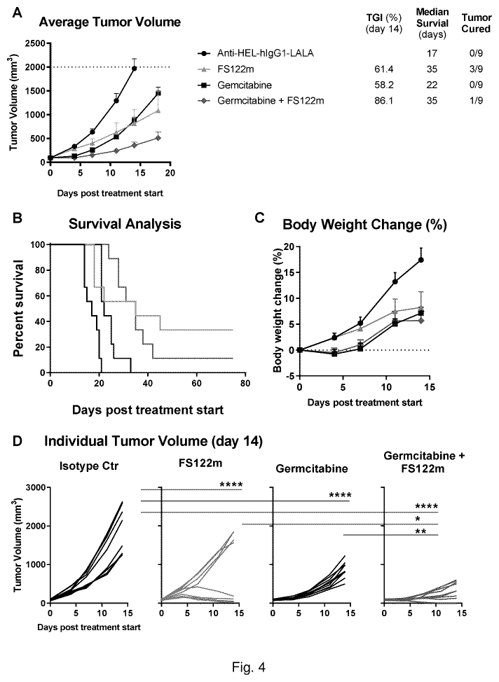

In CT26 colon tumor model in BALB/c mice, FS122m and gemcitabine monotherapies induced moderate TGI (61.4% and 58.2%, respectively) relative to the isotype control (P≤0.0001, Day 14), and prolonged median survival (35 and 22 days, respectively) relative to the isotype control (17 days) (FIGS. 4A, B and D). The combination of FS122m and gemcitabine resulted in greater TGI (86.1%) than gemcitabine monotherapy (P≤0.0001, Day 18) and FS122m monotherapy (P=0.0113, Day 18) (FIG. 4A). Combination therapy also prolonged median survival (35 days) (FIG. 4B) and induced complete tumor regression in 1 of 9 mice compared to complete tumor regression in 3 of 9 mice treated with FS122m monotherapy and no complete tumor regression in 9 mice treated with cisplatin monotherapy (FIG. 4D).

In both JC-tumor and CT26 colon tumor-bearing mice, treatment with a combination of FS122m and gemcitabine resulted in greater TGI than the TGI observed when mice were treated with either FS122m monotherapy or gemcitabine monotherapy. The difference compared with FS122m and gemcitabine monotherapy was particularly stark in the JC mouse tumor model, where the combination of FS122m and cisplatin resulted in complete tumor regression in 5 out of 10 mice compared with 1 out of 10 mice with FS122m monotherapy and no mice in the gemcitabine monotherapy group (FIG. 3D). Combination of FS122m and gemcitabine did not cause significant body weight loss in either mouse tumor model, suggesting that the combination treatment was well tolerated in the animals (FIGS. 3C and 4C).

### Sequence Listing

### Heavy Chain Annotations

i. In amino acid sequences of the heavy chain of mAb², the variable domain is shown in italics, CDRs according to IMGT are shown in bold italics, CDRs according to Kabat are shown in italics and underlined (therefore any overlapping IMGT and Kabat CDR sequences are shown in bold, italics and underlined), CH1 domains are underlined, hinge regions are doubly underlined, CH2 domains are shown in bold (and, where applicable, location of the LALA mutation is shown in bold and underlined), CH3 domains are shown in plain font, and modified regions of CH3 structural loops are underlined (no underlining if loop is unchanged).

ii. In amino acid sequences of variable domains, CDRs according to IMGT are shown in bold and italics, CDRs according to Kabat are shown in italics and underlined (therefore any overlapping IMGT and Kabat CDR sequences are shown in bold, italics and underlined).

iii. CDR amino acid sequences according to both IMGT and Kabat are provided.

### Light Chain Annotations

i. In the amino acid sequence of the light chain of mAb², variable domains are shown in italics, CDRs according to IMGT are shown in bold and italics, and CDRs according to Kabat are shown in italics and underlined (therefore any overlapping IMGT and Kabat CDR sequences are shown in bold, italics and underlined).

ii. In the amino acid sequence of the variable domain, CDRs according to IMGT are shown in bold and italics, and CDRs according to Kabat are shown in italics and underlined (therefore any overlapping IMGT and Kabat CDR sequences are shown in bold, italics and underlined).

iii. CDR amino acid sequences according to both IMGT and Kabat are provided.

Amino acid sequences of FS22-172-003-AA/FS28-256-271 mAb²
SEQ ID NO: 1
Heavy chain AA (without LALA)
EVQLLESGGGLVQPGGSLRLSCAASGFTFTHTYMSWVRQAPGKGLEWVSAI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
Heavy chain AA (with LALA)
SEQ ID NO: 2
EVQLLESGGGLVQPGGSLRLSCAASGFTFTHTYMSWVRQAPGKGLEWVSAI
SPTYSTTNYADSVKGRFTISRDNNKNTLYLQMNSLRAEDTAVYYCARYNAYHAALDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
VH domain AA
SEQ ID NO: 3
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
HCDR1 (AA) (IMGT)
SEQ ID NO: 4
GFTFTHTY
HCDR1 (AA) (Kabat)
SEQ ID NO: 5
HTYMS
HCDR2 (AA) (IMGT)
SEQ ID NO: 6
ISPTYSTT
HCDR2 (AA) Kabat)
SEQ ID NO: 7
AISPTYSTTNYADSVKG
HCDR3 (AA) (IMGT)
SEQ ID NO: 8
ARYNAYHAALDY
HCDR3 (AA) (Kabat)
SEQ ID NO: 9
YNAYHAALDY
Light chain AA
SEQ ID NO: 10
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 11
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 16
QQTVPYPYT
LCDR3 (AA) (Kabat)
SEQ ID NO: 16
QQTVPYPYT
Amino acid sequences of FS22-172-003-AA/FS28-024-052 mAb²
Heavy chain AA (without LALA)
SEQ ID NO: 17
EVQLLESGGGLVQPGGSLRLSCAASGFTLSYSSMSWVRQAPGKGLEWVSE!
TPSTGYTHYADSVKGRFTI
SRDNSKNTLYLQMNSLRAEDTAVYYCARRALLFDYWGQGTLVTVSSASTKGPSVFPLAPSSKSTSGGT
AALGCLVKDYFPEPVTVSWNSGALTSGVHTFPAVLQSSGLYSLSSVVTVPSSSLGTQTYICNVNHKPSNT
KVDKKVEPKSCDKTHTCPPCPAPELLGGPSVFLFPPKPKDTLMISRTPEVTCVVVDVSHEDPEVKFNWY
VDGVEVHNAKTKPREEQYNSTYRVVSVLTVLHQDWLNGKEYKCKVSNKALPAPIEKTISKAKGQPREP
QVYTLPPSRDELPYIIPPYNQVSLTCLVKGFYPSDIAVEWESNGQPENNYKTTPPVLDSDGSFFLYSKLTV
GADRWLEGNVFSCSVMHEALHNHYTQKSLSLSPG
Heavy chain AA (with LALA)
SEQ ID NO: 18
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALLFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
VH domain AA
SEQ ID NO: 19
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALLFDYWGQGTLVTVSS
HCDR1 (AA) (IMGT)
SEQ ID NO: 20
GFTLSYSS
HCDR1 (AA) (Kabat)
SEQ ID NO: 21
YSSMS
HCDR2 (AA) (IMGT)
SEQ ID NO: 22
ITPSTGYT
HCDR2 (AA) Kabat)
SEQ ID NO: 23
FITPSTGYTHYADSVKG
HCDR3 (AA) (IMGT)
SEQ ID NO: 24
ARRALLFDY
HCDR3 (AA) (Kabat)
SEQ ID NO: 25
RALLFDY
Light chain AA
SEQ ID NO: 26
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 27
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 28
QQASSYPLT
LCDR3 (AA) (Kabat)
SEQ ID NO: 28
QQASSYPLT
Amino acid sequences of FS22-172-003-AA/FS28-256-021 mAb²
Heavy chain AA (without LALA)
SEQ ID NO: 29
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 30
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 3
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
HCDR1 (AA) (IMGT)
SEQ ID NO: 4
GFTFTHTY
HCDR1 (AA) (Kabat)
SEQ ID NO: 5
HTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 8
ARYNAYHAALDY
HCDR3 (AA) (Kabat)
SEQ ID NO: 9
YNAYHAALDY
Light chain AA
SEQ ID NO: 32
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 33
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 34
QQHNQYPNT
LCDR3 (AA) (Kabat)
SEQ ID NO: 34
QQHNQYPNT
Amino acid sequences of FS22-172-003-AA/FS28-256-012 mAb²
Heavy chain AA (without LALA)
SEQ ID NO: 35
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 36
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 3
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
HCDR1 (AA) (IMGT)
SEQ ID NO: 4
GFTFTHTY
HCDR1 (AA) (Kabat)
SEQ ID NO: 5
HTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 8
ARYANYHAALDY
HCDR3 (AA) (Kabat)
SEQ ID NO: 9
YNAYHAALDY
Light chain AA
SEQ ID NO: 37
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
VL domain AA
SEQ ID NO: 38
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 39
QQSYYYPIT
LCDR3 (AA) (Kabat)
SEQ ID NO: 39
QQSYYYPIT
Amino acid sequences of FS22-172-003-AA/FS28-256-023 mAb²
Heavy chain AA (without LALA)
SEQ ID NO: 40
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
Heavy chain AA (with LALA)
SEQ ID NO: 41
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
VH domain AA
SEQ ID NO: 42
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSS
HCDR1 (AA) (IMGT)
SEQ ID NO: 43
GFTFTQTY
HCDR1 (AA) (Kabat)
SEQ ID NO: 44
QTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 45
ARYNAYQIGLDY
HCDR3 (AA) (Kabat)
SEQ ID NO: 46
YNAYQIGLDY
Light chain AA
SEQ ID NO: 32
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 33
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 34
QQHNQYPNT
LCDR3 (AA) (Kabat)
SEQ ID NO: 34
QQHNQYPNT
Amino acid sequences of FS22-172-003-AA/FS28-256-024 mAb²
Heavy chain AA (without LALA)
SEQ ID NO: 29
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 30
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 3
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
HCDR1 (AA) (IMGT)
SEQ ID NO: 4
GFTFTHTY
HCDR1 (AA) (Kabat)
SEQ ID NO: 5
HTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 8
ARYNAYHAALDY
HCDR3 (AA) (Kabat)
SEQ ID NO: 9
YNAYHAALDY
Light chain AA
SEQ ID NO: 47
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 48
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 49
QQALGYPHT
LCDR3 (AA) (IMGT)
SEQ ID NO: 49
QQALGYPHT
Amino acid sequences of FS22-172-003-AA/FS28-256-026 mAb2
Heavy chain AA (without LALA)
SEQ ID NO: 40
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
Heavy chain AA (with LALA)
SEQ ID NO: 41
EVQLLESGGGLVQPGGSLRLSCAASGFTFTQTYMSWVRQAPGKGLEWVSN
ISPTYSTTNYADSKVGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREQPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
VH domain AA
SEQ ID NO: 42
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSS
HCDR1 (AA) (IMGT)
SEQ ID NO: 43
GFTFTQTY
HCDR1 (AA) (Kabat)
SEQ ID NO: 44
QTYMS
HCDR2 (AA) (IMGT)
SEQ ID NO: 6
ISPTYSTT
HCDR2 (AA) (Kabat)
SEQ ID NO: 31
NISPTYSTTNYADSVKG
HCDR3 (AA) (IMGT)
SEQ ID NO: 45
ARYNAYQIGLDY
HCDR3 (AA) (Kabat)
SEQ ID NO: 46
YNAYQIGLDY
Light chain AA
SEQ ID NO: 47
EIVLTQSPGTLSLSPGERATLSCRASQSVSSSYLAWYQQKPGQAPRLLIYGA
SSRATGIPDRFSGSGSGTDFTLTISRLEPEDFACYYVQQALGYPHTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 48
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 49
QQALGYPHT
LCDR3 (AA) (Kabat)
SEQ ID NO: 49
QQALGYPHT
Amino acid sequences of FS22-172-003-AA/FS28-256-027 mAb²
Heavy chain AA (without LALA)
SEQ ID NO: 29
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 30
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 3
EVQLLESGGGLVQPGGSLRLSCAASGFTFTHTYMSWVRQAPGKGLEWVSN
ISPTYSTTNYADSVKGRFTISRDNNKNTLYLQMNSLRAEDTAVYYCARYNAYHAALDYWGQGTLVTVSS
HCDR1 (AA) (IMGT)
SEQ ID NO: 4
GFTFTHTY
HCDR1 (AA) (Kabat)
SEQ ID NO: 5
HTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 8
ARYNAYHAALDY
HCDR3 (AA) (Kabat)
SEQ ID NO: 9
YNAYHAALDY
Light chain AA
SEQ ID NO: 10
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 11
EIVLTQSPGTLSLSPGERATLSCRASQSVSSSYLAWYQQKPGQAPRLLIYGA
SSRATGIPDRFSGSGSGTDFTLTISRLEPEDFAVYYCQQTVPYPYTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 16
QQTVPYPYT
LCDR3 (AA) (Kabat)
SEQ ID NO: 16
QQTVPYPYT
Amino acid sequences of FS22-172-003-AA/FS28-256-001 mAb²
Heavy chain AA (without LALA)
SEQ ID NO: 50
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 51
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 52
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
HCDR1 (AA) (IMGT)
SEQ ID NO: 53
GFTFTETY
HCDR1 (AA) (Kabat)
SEQ ID NO: 54
ETYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 55
ARYNSYQGGLDY
HCDR3 (AA) (Kabat)
SEQ ID NO: 56
YNSYQGGLDY
Light chain AA
SEQ ID NO: 32
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 33
EIVLTQSPGTLSLSPGERATLSCRASQSVSSSYLAWYQQKPGQAPRLLIYGA
SSRATGIPDRFSGSGSGTDFTLTISRLEPEDFAVYYCQQHNQYPNTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 34
QQHNQYPNT
LCDR3 (AA) (Kabat)
SEQ ID NO: 34
QQHNQYPNT
Amino acid sequences of FS22-172-003-AA/FS28-256-005 mAb²
Heavy chain AA (without LALA)
SEQ ID NO: 50
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 51
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 52
EVQLLESGGGLVQPGGSLRLSCAASGFTFTETYMSWVRQAPGKGLEWVSNI
SPTYSTTNYADSVKGRFTISRDNSKNTLYLQMNSLRAEDTAVYYCARYNSYQGGLDYWGQGTLVTVSS
HCDR1 (AA) (IMGT)
SEQ ID NO: 53
GFTFTETY
HCDR1 (AA) (Kabat)
SEQ ID NO: 54
ETYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 55
ARYNSYQGGLDY
HCDR3 (AA) (Kabat)
SEQ ID NO: 56
YNSYQGGLDY
Light chain AA
SEQ ID NO: 47
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 48
EIVLTQSPGTLSLSPGERATLSCRASQSVSSSYLAWYQQKPGQAPRLLIYGA
SSRATGIPDRFSGSGSGTDFTLTISRLEPEDFAVYYCQQALGYPHTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 49
QQALGYPHT
LCDR3 (AA) (Kabat)
SEQ ID NO: 49
QQALGYPHT
Amino acid sequences of FS22-172-003-AA/FS28-256-014 mAb²
Heavy chain AA (without LALA)
SEQ ID NO: 57
EVQLLESGGGLVQPGGSLRLSCAASGFTFTDTYMSWVRQAPGKGLEWVSN
ISPTYSTTNYADSVKGRFTISRDNSKNTLYLQMNSLRAEDTAVYYCARYNAYAAGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 58
EVQLLESGGGLVQPGGSLRLSCAASGFTFTDTYMSWVRQAPGKGLEWVSN
ISPTYSTTNYADSVKGRFTISRDNSKNTLYLQMNSLRAEDTAVYYCARYNAYAAGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 59
EVQLLESGGGLVQPGGSLRLSCAASGFTFTDTYMSWVRQAPGKGLEWVSN
ISPTYSTTNYADSVKGRFTISRDNSKNTLYLQMNSLRAEDTAVYYCARYNAYAAGLDYWGQGTLVTVSS
HCDR1 (AA) (IMGT)
SEQ ID NO: 60
GFTFTDTY
HCDR1 (AA) (Kabat)
SEQ ID NO: 61
DTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 62
ARYNAYAAGLDY
HCDR3 (AA) (Kabat)
SEQ ID NO: 63
YNAYAAGLDY
Light chain AA
SEQ ID NO: 37
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
VL domain AA
SEQ ID NO: 38
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 39
QQSYYYPIT
LCDR3 (AA) (Kabat)
SEQ ID NO: 39
QQSYYYPIT
Amino acid sequences of FS22-172-003-AA/FS28-256-018 mAb²
Heavy chain AA (without LALA)
SEQ ID NO: 40
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPE
VTCVVVDVSHEDPEVKFNWYVDGVEVHNAKTKPREEQYNSTYRVVSVLTVLHQDWLNGKEYKCKVSN
KALPAPIEKTISKAKGQPREPQVYTLPPSRDELPYIIPPYNQVSLTCLVKGFYPSDIAVEWESNGQPENNY
KTTPPVLDSDGSFFLYSKLTVGADRWLEGNVFSCSVMHEALHNHYTQKSLSLSPG
Heavy chain AA (with LALA)
SEQ ID NO: 41
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSSA
STKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPEAAGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
VH domain AA
SEQ ID NO: 42
EVQLLESGGGLVQPGGSLRLSCAASGFTFTQTYMSWVRQAPGKGLEWVSN
ISPTYSTTNYADSVKGRFTISRDNSKNTLYLQMNSLRAEDTAVYYCARYNAYQIGLDYWGQGTLVTVSS
HCDR1 (AA) (IMGT)
SEQ ID NO: 43
GFTFTQTY
HCDR1 (AA) (Kabat)
SEQ ID NO: 44
QTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 45
ARYNAYQIGLDY
HCDR3 (AA) (Kabat)
SEQ ID NO: 46
YNAYQIGLDY
Light chain AA
SEQ ID NO: 37
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
VL domain AA
SEQ ID NO: 38
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 39
QQSYYYPIT
LCDR3 (AA) (Kabat)
SEQ ID NO: 39
QQSYYYPIT
Amino acid sequences of FS22-172-003-AA/FS28-256 mAb²
Heavy chain AA (without LALA)
SEQ ID NO: 64
EVQLLESGGGLVQPGGSLRLSCAASGFTFTNTYMSWVRQAPGKGLEWVSN
ISPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELLGGPSVFLFPPKPKDTLMISRTP
EVTCVVVDVSHEDPEVKFNWYVDGVEVHNAKTKPREEQYNSTYRVVSVLTVLHQDWLNGKEYKCKVS
NKALPAPIEKTISKAKGQPREPQVYTLPPSRDELPYIIPPYNQVSLTCLVKGFYPSDIAVEWESNGQPENN
YKTTPPVLDSDGSFFLYSKLTVGADRWLEGNVFSCSVMHEALHNHYTQKSLSLSPG
Heavy chain AA (with LALA)
SEQ ID NO: 65
EVQLLESGGGLVQPGGSLRLSCAASGFTFTNTYMSWVRQAPGKGLEWVSN
ISPTYSTTNYADSVKGRFTISRDNSKNTLYLQMNSLRAEDTAVYYCARYNSYQGGLDYWGQGTLVTVSS
ASTKGPSVFPLAPSSKSTSGGTAALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPEAAGGPSVFLFPPKPKDTLMISRT
PEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELPYIIPPYNQVSLTCLVKGFYPSDIAVEWESNGQPEN
NYKTTPPVLDSDGSFFLYSKLTVGADRWLEGNVFSCSVMHEALHNHYTQKSLSLSPG
VH domain AA
SEQ ID NO: 66
EVQLLESGGGLVQPGGSLRLSCAASGFTFTNTYMSWVRQAPGKGLEWVSN
ISPTYSTTNYADSVKGRFTISRDNSKNTLYLQMNSLRAEDTAVYYCARYNSYQGGLDYWGQGTLVTVSS
HCDR1 (AA) (IMGT)
SEQ ID NO: 67
GFTFTNTY
HCDR1 (AA) (Kabat)
SEQ ID NO: 68
NTYMS
HCDR2 (AA) (IMGT)
SEQ ID NO: 6
ISPTYSTT
HCDR2 (AA) Kabat)
SEQ ID NO: 31
NISPTYSTTNYADSVKG
HCDR3 (AA) (IMGT)
SEQ ID NO: 55
ARYNSYQGGLDY
HCDR3 (AA) (Kabat)
SEQ ID NO: 56
YNSYQGGLDY
Light chain AA
SEQ ID NO: 37
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIKRTVAAPSVFIFPPSDE
QLKSGTASVVCLLNNFYPREAKVQWKVDNALQSGNSQESVTEQDSKDSTYSLSSTLTLSKADYEKHKVY
ACEVTHQGLSSPVTKSFNRGEC
VL domain AA
SEQ ID NO: 38
EIVLTQSPGTLSLSPGERATLSCRASQSVSSSYLAWYQQKPGQAPRLLIYGA
SSRATGIPDRFSGSGSGTDFTLTISRLEPEDFAVYYCQQSYYYPITFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 39
QQSYYYPIT
LCDR3 (AA) (Kabat)
SEQ ID NO: 39
QQSYYYPIT
Amino acid sequences of FS22-172-003-AA/FS28-024-051 mAb²
Heavy chain AA (without LALA)
SEQ ID NO: 69
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALIFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
Heavy chain AA (with LALA)
SEQ ID NO: 70
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALIFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
VH domain AA
SEQ ID NO: 71
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALIFDYWGQGTLVTVSS
HCDR1 (AA) (IMGT)
SEQ ID NO: 21
GFTLSYSS
HCDR1 (AA) (Kabat)
SEQ ID NO: 22
YSSMS
HCDR2 (AA) (IMGT)
SEQ ID NO: 23
ITPSTGYT
HCDR2 (AA) Kabat)
SEQ ID NO: 24
FITPSTGYTHYADSVKG
HCDR3 (AA) (IMGT)
SEQ ID NO: 72
ARRALIFDY
HCDR3 (AA) (Kabat)
SEQ ID NO: 73
RALIFDY
Light chain AA
SEQ ID NO: 26
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 27
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 28
QQASSYPLT
LCDR3 (AA) (Kabat)
SEQ ID NO: 28
QQASSYPLT
Amino acid sequences of FS22-172-003-AA/FS28-024-053 mAb2
Heavy chain AA (without LALA)
SEQ ID NO: 74
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALVFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
Heavy chain AA (with LALA)
SEQ ID NO: 75
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALVFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
VH domain AA
SEQ ID NO: 76
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALVFDYWGQGTLVTVSS
HCDR1 (AA) (IMGT)
SEQ ID NO: 21
GFTLSYSS
HCDR1 (AA) (Kabat)
SEQ ID NO: 22
YSSMS
HCDR2 (AA) (IMGT)
SEQ ID NO: 23
ITPSTGYT
HCDR2 (AA) (Kabat)
SEQ ID NO: 77
ARRALVFDY
HCDR3 (AA) (Kabat)
SEQ ID NO: 78
RALVFDY
Light chain AA
SEQ ID NO: 26
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 27
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
SEQ ID NO: 15
LCDR2 (AA) (Kabat)
GASSRAT
LCDR3 (AA) (IMGT)
SEQ ID NO: 28
QQASSYPLT
LCDR3 (AA) (Kabat)
SEQ ID NO: 28
QQASSYPLT
Amino acid sequences of FS22-172-003-AA/FS28-024 mAb²
Heavy chain AA (without LALA)
SEQ ID NO: 79
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALTFDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
Heavy chain AA (with LALA)
SEQ ID NO: 80
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALTEDYWGQGTLVTVSSASTK
GPSVFPLAPSSKSTSGGTAALGCLVKDYFPEPVTVSWNSGALTSGVHTFPAVLQSSGLYSLSSVVTVPS
SSLGTQTYICNVNHKPSNTKVDKKVEPKSCDKTHTCPPCPAPEAAGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDELPYIIPPYNQVSLTCLVKGFYPSDIAVEWESNGQPENNYKT
TPPVLDSDGSFFLYSKLTVGADRWLEGNVFSCSVMHEALHNHYTQKSLSLSPG
VH domain AA
SEQ ID NO: 81
EVQLLESGGGLVQPGGSLRLSCAASGFTLSYSSMSWVRQAPGKGLEWVSFI
TPSTGYTHYADSVKGRFTISRDNSKNTLYLQMNSLRAEDTAVYYCARRALTFDYWGQGTLVTVSS
HCDR1 (AA) (IMGT)
SEQ ID NO: 21
GFTLSYSS
HCDR1 (AA) (Kabat)
SEQ ID NO: 22
YSSMS
HCDR2 (AA) (IMGT)
SEQ ID NO: 23
ITPSTGYT
HCDR2 (AA) Kabat)
SEQ ID NO: 24
FITPSTGYTHYADSVKG
HCDR3 (AA) (IMGT)
SEQ ID NO: 82
ARRALTFDY
HCDR3 (AA) (Kabat)
SEQ ID NO: 83
RALTFDY
Light chain AA
SEQ ID NO: 26
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIKRTVAAPSVFIFPPSD
EQLKSGTASVVCLLNNFYPREAKVQWKVDNALQSGNSQESVTEQDSKDSTYSLSSTLTLSKADYEKHKV
YACEVTHQGLSSPVTKSFNRGEC
VL domain AA
SEQ ID NO: 27
EIVLTQSPGTLSLSPGERATLSCRASQSVSSSYLAWYQQKPGQAPRLLIYGA
SSRATGIPDRFSGSGSGTDFTLTISRLEPEDFAVYYCQQASSYPLTFGQGTKVEIK
LCDR1 (AA) (IMGT)
SEQ ID NO: 12
QSVSSSY
LCDR1 (AA) (Kabat)
SEQ ID NO: 13
RASQSVSSSYLA
LCDR2 (AA) (IMGT)
SEQ ID NO: 14
GAS
LCDR2 (AA) (Kabat)
SEQ ID NO: 15
GASSRAT
LCDR3 (AA) (IMGT)
SEQ ID NO: 28
QQASSYPLT
LCDR3 (AA) (Kabat)
SEQ ID NO: 28
QQASSYPLT
Amino acid sequences of FS122m (surrogate anti-mMSLN mCD137 Fcab G1-AA):
Heavy chain AA (with LALA)
SEQ ID NO: 84
EVQLLESGGGLVQPGGSLRLSCAASGFTFSTYFMVWVRQAPGKGLEWVSMIS
PKSSNTYYADSVKGRFTISRDNSKNTLYLQMNSLRAEDTAVYYCARYHISPRFDYWGQGTLVTVSSAST
KGPSVFPLAPSSKSTSGGTAALGCLVKDYFPEPVTVSWNSGALTSGVHTFPAVLQSSGLYSLSSVVTVP
SSSLGTQTYICNVNHKPSNTKVDKKVEPKSCDKTHTCPPCPAPEAAGGPSVFLFPPKPKDTLMISRTPEV
TCVVVDVSHEDPEVKFNWYVDGVEVHNAKTKPREEQYNSTYRVVSVLTVLHQDWLNGKEYKCKVSNKA
LPAPIEKTISKAKGQPREPQVYTLPPSRDEPYWSYVSLTCLVKGFYPSDIAVEWESNGQPENNYKTTPPV
LDSDGSFFLYSKLTVMNYRWELGNVFSCSVMHEALHNHYTQKSLSLSPGK
Light chain AA
SEQ ID NO: 85
EIVLTQSPGTLSLSPGERATLSCRASQSVSSSYLAWYQQKPGQAPRLLIYGASS
RATGIPDRFSGSGSGTDFTLTISRLEPEDFAVYYCQQPFPFSFTFGQGTKVEIKRTVAAPSVFIFPPSDEQ
LKSGTASVVCLLNNFYPREAKVQWKVDNALQSGNSQESVTEQDSKDSTYSLSSTLTLSKADYEKHKVYA
CEVTHQGLSSPVTKSFNRGEC
Amino acid sequences of CH3 domain and amino acid sequence of modified regions of CH3 AB and EF
structural loops of all FS22-172-003 Fcab-containing mAb² clones and the FS22-172-003 Fcab
CH3 domain
SEQ ID NO: 86
GQPREPQVYTLPPSRDELPYIIPPYNQVSLTCLVKGFYPSDIAVEWESNGQP
ENNYKTTPPVLDSDGSFFLYSKLTVGADRWLEGNVFSCSVMHEALHNHYTQKSLSLSPG
AB Loop
SEQ ID NO: 87
PYIIPPY
EF Loop
SEQ ID NO: 88
GADRWLE
CD Loop
SEQ ID NO: 89
SNGQPENNY
Amino acid sequences of CH2 domain containing LALA mutation, PA mutation or LALA-PA mutation
(mutations in bold and underlined)
CH2 (LALA)
SEQ ID NO: 90
APEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAPIEKTISKAK
CH2 (PA)
SEQ ID NO: 91
APELLGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALAAPIEKTISKAK
CH2 (LALA-PA)
SEQ ID NO: 92
APEAAGGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALAAPIEKTISKAK

### REFERENCES

A number of publications are cited above in order to more fully describe and disclose the invention and the state of the art to which the invention pertains. Full citations for these references are provided below. The entirety of each of these references is incorporated herein.
1 Bartkowiak, T. & Curran, M. A. 4-1BB Agonists: Multi-Potent Potentiators of Tumor Immunity. Front Oncol 5, 117, doi:10.3389/fonc.2015.00117 (2015).
2 Compte, M. et al. A tumor-targeted trimeric 4-1BB-agonistic antibody induces potent anti-tumor immunity without systemic toxicity. Nat Commun 9, 4809, doi:10.1038/s41467-018-07195-w (2018).
3 Kataoka, K. et al. Integrated molecular analysis of adult T cell leukemia/lymphoma. Nat Genet 47, 1304-1315, doi:10.1038/ng.3415 (2015).
4 Sanmamed, M. F. et al. Agonists of Co-stimulation in Cancer Immunotherapy Directed Against CD137, OX40, GITR, CD27, CD28, and ICOS. Semin Oncol 42, 640-655, doi:10.1053/j.seminoncol.2015.05.014 (2015).
5 Yonezawa, A., Chester, C., Rajasekaran, N. & Kohrt, H. E. Harnessing the innate immune system to treat cancer: enhancement of antibody-dependent cellular cytotoxicity with anti-CD137 Ab. Chin Clin Oncol 5, 5, doi:10.3978/j.issn.2304-3865.2016.02.05 (2016).
6 Yonezawa, A., Dutt, S., Chester, C., Kim, J. & Kohrt, H. E. Boosting Cancer Immunotherapy with Anti-CD137 Antibody Therapy. Clin Cancer Res 21, 3113-3120, doi:10.1158/1078-0432.CCR-15-0263 (2015).
7 Melero, I., Johnston, J. V., Shufford, W. W., Mittler, R. S. & Chen, L. NK1.1 cells express 4-1BB (CDw137) costimulatory molecule and are required for tumor immunity elicited by anti-4-1BB monoclonal antibodies. Cell Immunol 190, 167-172, doi:10.1006/cimm.1998.1396 (1998).
8 Vinay, D. S. & Kwon, B. S. Role of 4-1BB in immune responses. Semin Immunol 10, 481-489, doi:10.1006/smim.1998.0157 (1998).
9 Cannons, J. L., Hoeflich, K. P., Woodgett, J. R. & Watts, T. H. Role of the stress kinase pathway in signaling via the T cell costimulatory receptor 4-1BB. J Immunol 163, 2990-2998 (1999).
10 Sica, G. & Chen, L. Biochemical and immunological characteristics of 4-1BB (CD137) receptor and ligand and potential applications in cancer therapy. Arch Immunol Ther Exp (Warsz) 47, 275-279 (1999).
11 Lee, H. W. et al. 4-1BB promotes the survival of CD8+ T lymphocytes by increasing expression of Bcl-xL and Bfl-1. J Immunol 169, 4882-4888, doi:10.4049/jimmunol. 169.9.4882 (2002).
12 Lee, H. W., Nam, K. O., Park, S. J. & Kwon, B. S. 4-1BB enhances CD8+ T cell expansion by regulating cell cycle progression through changes in expression of cyclins D and E and cyclin-dependent kinase inhibitor p27kip1. Eur J Immunol 33, 2133-2141, doi:10.1002/eji.200323996 (2003).
13 Chacon, J. A. et al. Co-stimulation through 4-1BB/CD137 improves the expansion and function of CD8 (+) melanoma tumor-infiltrating lymphocytes for adoptive T-cell therapy. PLOS One 8, e60031, doi:10.1371/journal.pone.0060031 (2013).
14 Fisher, T. S. et al. Targeting of 4-1BB by monoclonal antibody PF-05082566 enhances T-cell function and promotes anti-tumor activity. Cancer Immunol Immunother 61, 1721-1733, doi:10.1007/s00262-012-1237-1 (2012).
15 Claus, C. et al. Tumor-targeted 4-1BB agonists for combination with T cell bispecific antibodies as off-the-shelf therapy. Sci Transl Med 11, doi:10.1126/scitranslmed.aav5989 (2019).
16 Qi, X. et al. Optimization of 4-1BB antibody for cancer immunotherapy by balancing agonistic strength with FcgammaR affinity. Nat Commun 10, 2141, doi:10.1038/s41467-019-10088-1 (2019).
17 Azpilikueta, A. et al. Successful Immunotherapy against a Transplantable Mouse Squamous Lung Carcinoma with Anti-PD-1 and Anti-CD137 Monoclonal Antibodies. J Thorac Oncol 11, 524-536, doi:10.1016/j.jtho.2016.01.013 (2016).
18 Narazaki, H., Zhu, Y., Luo, L., Zhu, G. & Chen, L. CD137 agonist antibody prevents cancer recurrence: contribution of CD137 on both hematopoietic and nonhematopoietic cells. Blood 115, 1941-1948, doi:10.1182/blood-2008-12-192591 (2010).
19 Wilcox, R. A. et al. Provision of antigen and CD137 signaling breaks immunological ignorance, promoting regression of poorly immunogenic tumors. J Clin Invest 109, 651-659, doi:10.1172/JCI14184 (2002).
20 Weigelin, B. et al. Focusing and sustaining the antitumor CTL effector killer response by agonist anti-CD137 mAb. Proc Natl Acad Sci USA 112, 7551-7556, doi:10.1073/pnas. 1506357112 (2015).
21 Segal, N. H. et al. Results from an Integrated Safety Analysis of Urelumab, an Agonist Anti-CD137 Monoclonal Antibody. Clin Cancer Res 23, 1929-1936, doi:10.1158/1078-0432.CCR-16-1272 (2017).
22 Timmerman, J. et al. Urelumab alone or in combination with rituximab in patients with relapsed or refractory B-cell lymphoma. Am J Hematol 95, 510-520, doi:10.1002/ajh.25757 (2020).
23 Chin, S. M. et al. Structure of the 4-1BB/4-1BBL complex and distinct binding and functional properties of utomilumab and urelumab. Nat Commun 9, 4679, doi:10.1038/s41467-018-07136-7 (2018).
24 Eskiocak, U. et al. Differentiated agonistic antibody targeting CD137 eradicates large tumors without hepatotoxicity. JCI Insight 5, doi:10.1172/jci.insight. 133647 (2020).
25 Trub, M. et al. Fibroblast activation protein-targeted-4-1BB ligand agonist amplifies effector functions of intratumoral T cells in human cancer. J Immunother Cancer 8, doi:10.1136/jitc-2019-000238 (2020).
26 Mardiana, S. et al. A Multifunctional Role for Adjuvant Anti-4-1BB Therapy in Augmenting Antitumor Response by Chimeric Antigen Receptor T Cells. Cancer Res 77, 1296-1309, doi:10.1158/0008-5472.CAN-16-1831 (2017).
27 Ordóñez, N. G. Application of mesothelin immunostaining in tumor diagnosis. Am J Surg Pathol 27, 1418-1428, doi:10.1097/00000478-200311000-00003 (2003).
28 Chang, K. & Pastan, I. Molecular cloning of mesothelin, a differentiation antigen present on mesothelium, mesotheliomas, and ovarian cancers. Proc Natl Acad Sci USA 93, 136-140, doi:10.1073/pnas.93.1.136 (1996).
29 Miettinen, M. & Sarlomo-Rikala, M. Expression of calretinin, thrombomodulin, keratin 5, and mesothelin in lung carcinomas of different types: an immunohistochemical analysis of 596 tumors in comparison with epithelioid mesotheliomas of the pleura. Am J Surg Pathol 27, 150-158, doi:10.1097/00000478-200302000-00002 (2003).
30 Hassan, R. et al. Anti-tumor activity of K1-LysPE38QQR, an immunotoxin targeting mesothelin, a cell-surface antigen overexpressed in ovarian cancer and malignant mesothelioma. J Immunother 23, 473-479, doi:10.1097/00002371-200007000-00011 (2000).
31 Hassan, R. et al. Phase 1 study of the immunotoxin LMB-100 in patients with mesothelioma and other solid tumors expressing mesothelin. Cancer 126, 4936-4947, doi:10.1002/cncr.33145 (2020).
32 Hassan, R. et al. Clinical Response of Live-Attenuated, Listeria monocytogenes Expressing Mesothelin (CRS-207) with Chemotherapy in Patients with Malignant Pleural Mesothelioma. Clin Cancer Res 25, 5787-5798, doi:10.1158/1078-0432.CCR-19-0070 (2019).
33 Hassan, R. et al. First-in-Human, Multicenter, Phase I Dose-Escalation and Expansion Study of Anti-Mesothelin Antibody-Drug Conjugate Anetumab Ravtansine in Advanced or Metastatic Solid Tumors. J Clin Oncol 38, 1824-1835, doi:10.1200/JCO.19.02085 (2020).
34 Hassan, R. et al. Mesothelin Immunotherapy for Cancer: Ready for Prime Time? J Clin Oncol 34, 4171-4179, doi:10.1200/JCO.2016.68.3672 (2016).
35 Hassan, R. et al. Inhibition of mesothelin-CA-125 interaction in patients with mesothelioma by the anti-mesothelin monoclonal antibody MORAb-009: Implications for cancer therapy. Lung Cancer 68, 455-459, doi:10.1016/j.lungcan.2009.07.016 (2010).
36 Kreitman, R. J., Hassan, R., Fitzgerald, D. J. & Pastan, I. Phase I trial of continuous infusion anti-mesothelin recombinant immunotoxin SS1P. Clin Cancer Res 15, 5274-5279, doi:10.1158/1078-0432.CCR-09-0062 (2009).
37 Hassan, R. et al. Phase I study of SS1P, a recombinant anti-mesothelin immunotoxin given as a bolus I.V. infusion to patients with mesothelin-expressing mesothelioma, ovarian, and pancreatic cancers. Clin Cancer Res 13, 5144-5149, doi:10.1158/1078-0432.CCR-07-0869 (2007).
38 Ho, M. et al. Mesothelin expression in human lung cancer. Clin Cancer Res 13, 1571-1575, doi:10.1158/1078-0432.CCR-06-2161 (2007).
39 Kelly, R. J., Sharon, E., Pastan, I. & Hassan, R. Mesothelin-targeted agents in clinical trials and in preclinical development. Mol Cancer Ther 11, 517-525, doi:10.1158/1535-7163.MCT-11-0454 (2012).
40 Fucikova, J. et al. Detection of immunogenic cell death and its relevance for cancer therapy. Cell Death Dis 11, 1013, doi:10.1038/s41419-020-03221-2 (2020).
41 Vanmeerbeek, I. et al. Trial watch: chemotherapy-induced immunogenic cell death in immuno-oncology. Oncoimmunology 9, 1703449, doi:10.1080/2162402x.2019.1703449 (2020).
42 Wang, Q., et al., Immunogenic cell death in anticancer chemotherapy and its impact on clinical studies. Cancer Lett 438, 17-23 (2018).
43 Bracci, L., et al., Immune-based mechanisms of cytotoxic chemotherapy: implications for the design of novel and rationale-based combined treatments against cancer. Cell Death Differ 21(1), 15-25 (2014).
44 Vincent, J., et al., 5-Fluorouracil selectively kills tumor-associated myeloid-derived suppressor cells resulting in enhanced T cell-dependent antitumor immunity. Cancer Res 70(8), 3052-61 (2010).
45 Welters, M. J., et al., Vaccination during myeloid cell depletion by cancer chemotherapy fosters robust T cell responses. Sci Transl Med 8(334), 334-352 (2016).
46 Gattinoni, L., et al., Removal of homeostatic cytokine sinks by lymphodepletion enhances the efficacy of adoptively transferred tumor-specific CD8+ T cells. J Exp Med 202(7), 907-12 (2005).
47 Tallarida R J. Quantitative methods for assessing drug synergism. Genes Cancer 2(11): 1003-8. doi:10.1177/1947601912440575 (2011).
For standard molecular biology techniques, see Sambrook, J., Russel, D. W. Molecular Cloning, A Laboratory Manual. 3 ed. 2001, Cold Spring Harbor, New York: Cold Spring Harbor Laboratory Press

1. An antibody molecule that binds MSLN and CD137.

2. A method of treating cancer in an individual, the method comprising: administering to the individual a therapeutically effective amount of an antibody molecule that binds MSLN and CD137 and a chemotherapeutic.

3. A kit comprising:
(a) an antibody molecule that binds MSLN and CD137 and a pharmaceutically acceptable excipient; and
(b) a chemotherapeutic and a pharmaceutically acceptable excipient.

4. The method according to claim 2, wherein the antibody molecule that binds MSLN and CD137 comprises
(a) a complementary determining region (CDR)-based antigen-binding site for MSLN; and
(b) a CD137 antigen-binding site located in a CH3 domain of the antibody molecule.

5. The method according to claim 4, wherein the CDR-based antigen-binding site for MSLN comprises CDRs 1-6 set forth in:
(i) SEQ ID NOs 4, 6, 8, 12, 14, and 16, respectively [FS28-256-271];
(ii) SEQ ID NOs 20, 22, 24, 12, 14 and 28, respectively [FS28-024-052];
(iii) SEQ ID NOs 4, 6, 8, 12, 14 and 34, respectively [FS28-256-021];
(iv) SEQ ID NOs 4, 6, 8, 12, 14, and 39, respectively [FS28-256-012];
(v) SEQ ID NOs 43, 6, 45, 12, 14 and 34, respectively [FS28-256-023];
(vi) SEQ ID NOs 4, 6, 8, 12, 14 and 49, respectively [FS28-256-024];
(vii) SEQ ID NOs 43, 6, 45, 12, 14 and 49, respectively [FS28-256-026];
(viii) SEQ ID NOs 4, 6, 8, 12, 14 and 16, respectively [FS28-256-027];
(ix) SEQ ID NOs 53, 6, 55, 12, 14 and 34, respectively [FS28-256-001];
(x) SEQ ID NOs 53, 6, 55, 12, 14 and 49, respectively [FS28-256-005];
(xi) SEQ ID NOs 60, 6, 62, 12, 14 and 39, respectively [FS28-256-014];
(xii) SEQ ID NOs 43, 6, 45, 12, 14 and 39, respectively [FS28-256-018];
(xiii) SEQ ID NOs 67, 6, 55, 12, 14 and 39, respectively [FS28-256];
(xiv) SEQ ID NOs 21, 23, 72, 12, 14 and 28, respectively [FS28-024-051];
(xv) SEQ ID NOs 21, 23, 77, 12, 14 and 28, respectively [FS28-024-053]; or
(xvi) SEQ ID NOs 21, 23, 82, 12, 14 and 28, respectively [FS28-024]; and wherein the CD137 antigen-binding site comprises a first sequence and a second sequence located in the AB and EF structural loops of the CH3 domain, respectively, wherein the first and second sequence have the sequence set forth in SEQ ID NOs 87 and 88 [FS22-172-003], respectively.

6. The method according to claim 5, wherein
(i) the first sequence is located between positions 14 and 17 of the CH3 domain of the antibody molecule; and/or
(ii) wherein the second sequence is located between 91 and 99 of the CH3 domain of the antibody molecule; and
wherein the amino acid residue numbering is according to the IMGT numbering scheme.

7. The method according to claim 2, wherein the antibody molecule comprises the CH3 domain sequence set forth in SEQ ID NO: 86 [FS22-172-003].

8. The method according to claim 2, wherein the antibody molecule comprises the heavy chain and light chain of antibody:
(i) FS22-172-003-AA/FS28-256-271 set forth in SEQ ID NOs 2 and 10, respectively;
(ii) FS22-172-003-AA/FS28-024-052 set forth in SEQ ID NOs 18 and 26, respectively;
(iii) FS22-172-003-AA/FS28-256-021 set forth in SEQ ID NOs 30 and 32, respectively;
(iv) FS22-172-003-AA/FS28-256-012 set forth in SEQ ID NOs 36 and 37, respectively;
(v) FS22-172-003-AA/FS28-256-023 set forth in SEQ ID NOs 41 and 32, respectively;
(vi) FS22-172-003-AA/FS28-256-024 set forth in SEQ ID NOs 30 and 47, respectively;
(vii) FS22-172-003-AA/FS28-256-026 set forth in SEQ ID NOs 41 and 47, respectively;
(viii) FS22-172-003-AA/FS28-256-027 set forth in SEQ ID NOs 30 and 10, respectively;
(ix) FS22-172-003-AA/FS28-256-001 set forth in SEQ ID NOs 51 and 32, respectively;
(x) FS22-172-003-AA/FS28-256-005 set forth in SEQ ID NOs 51 and 47, respectively;
(xi) FS22-172-003-AA/FS28-256-014 set forth in SEQ ID NOs 58 and 37, respectively;
(xii) FS22-172-003-AA/FS28-256-018 set forth in SEQ ID NOs 41 and 37, respectively;
(xiii) FS22-172-003-AA/FS28-256 set forth in SEQ ID NOs 65 and 37, respectively;
(xiv) FS22-172-003-AA/FS28-024-051 set forth in SEQ ID NOs 70 and 26, respectively;
(xv) FS22-172-003-AA/FS28-024-053 set forth in SEQ ID NOs 75 and 26, respectively; or
(xvi) FS22-172-003-AA/FS28-024 set forth in SEQ ID NOs 80 and 26, respectively.

9. The method according to claim 8, wherein the antibody molecule comprises the heavy chain sequence set forth in SEQ ID NO: 2 and the light chain sequence set forth in SEQ ID NO: 10 [FS22-172-003-AA/FS28-256-271].

10. The method according to claim 2, wherein the chemotherapeutic is an alkylating agent or an antimetabolite.

11. The method according to claim 10, wherein the alkylating agent is selected from altretamine, bendamustine, busulfan, carboplatin, carmustine, chlorambucil, cisplatin, cyclophosphamide (CPA), dacarbazine, ifosfamide, lomustine, mechlorethamine, melphalan, oxaliplatin, temozolomide, thiotepa, and trabectedin.

12. The method according to claim 10, wherein the antimetabolite is selected azacitidine, 5-fluorouracil (5-FU), 6-mercaptopurine (6-MP), capecitabine, cladribine, clofarabine, cytarabine, decitabine, floxuridine, fludarabine, gemcitabine, hydroxyurea, methotrexate, nelarabine, pemetrexed, pentostatin, pralatrexate, thioguanine, and a trifluridine/tipiracil combination.

13. The method according to claim 2, wherein the cancer expresses MSLN or has been determined to express MSLN and is selected from ovarian cancer, pancreatic adenocarcinoma, mesothelioma, cervical carcinoma, and non-small cell lung carcinoma.

14. The method according to claim 2, wherein treatment with the antibody molecule that binds MSLN and CD137 and the chemotherapeutic results in a greater anti-tumor effect than the combined anti-tumor effect when patients are treated with the antibody molecule that binds MSLN and CD137, or the chemotherapeutic, alone.

15. The method according to claim 14, wherein the anti-tumor effect is tumor growth inhibition, tumor volume reduction, increase in median survival, and/or increase in the percentage of patients experiencing complete tumor regression.

16. The method according to claim 2, wherein the antibody molecule that binds MSLN and CD137 and the chemotherapeutic are administered to the patient concomitantly or sequentially.

17. The method according to claim 2, wherein the method comprises determining whether the cancer expresses MSLN and treating the individual if the cancer expresses MSLN.

18. The antibody molecule according to claim 1, wherein the antibody molecule that binds MSLN and CD137 comprises
(a) a complementary determining region (CDR)-based antigen-binding site for MSLN; and
(b) a CD137 antigen-binding site located in a CH3 domain of the antibody molecule.

19. The antibody molecule according to claim 18, wherein the CDR-based antigen-binding site for MSLN comprises CDRs 1-6 set forth in:
(i) SEQ ID NOs 4, 6, 8, 12, 14, and 16, respectively [FS28-256-271];
(ii) SEQ ID NOs 20, 22, 24, 12, 14 and 28, respectively [FS28-024-052];
(iii) SEQ ID NOs 4, 6, 8, 12, 14 and 34, respectively [FS28-256-021];
(iv) SEQ ID NOs 4, 6, 8, 12, 14, and 39, respectively [FS28-256-012];
(v) SEQ ID NOs 43, 6, 45, 12, 14 and 34, respectively [FS28-256-023];
(vi) SEQ ID NOs 4, 6, 8, 12, 14 and 49, respectively [FS28-256-024];
(vii) SEQ ID NOs 43, 6, 45, 12, 14 and 49, respectively [FS28-256-026];
(viii) SEQ ID NOs 4, 6, 8, 12, 14 and 16, respectively [FS28-256-027];
(ix) SEQ ID NOs 53, 6, 55, 12, 14 and 34, respectively [FS28-256-001];
(x) SEQ ID NOs 53, 6, 55, 12, 14 and 49, respectively [FS28-256-005];
(xi) SEQ ID NOs 60, 6, 62, 12, 14 and 39, respectively [FS28-256-014];
(xii) SEQ ID NOs 43, 6, 45, 12, 14 and 39, respectively [FS28-256-018];
(xiii) SEQ ID NOs 67, 6, 55, 12, 14 and 39, respectively [FS28-256];
(xiv) SEQ ID NOs 21, 23, 72, 12, 14 and 28, respectively [FS28-024-051];
(xv) SEQ ID NOs 21, 23, 77, 12, 14 and 28, respectively [FS28-024-053]; or
(xvi) SEQ ID NOs 21, 23, 82, 12, 14 and 28, respectively [FS28-024]; and wherein the CD137 antigen-binding site comprises a first sequence and a second sequence located in the AB and EF structural loops of the CH3 domain, respectively, wherein the first and second sequence have the sequence set forth in SEQ ID NOs 87 and 88 [FS22-172-003], respectively.

20. The antibody molecule according to claim 2, wherein the antibody molecule comprises the heavy chain and light chain of antibody:
(i) FS22-172-003-AA/FS28-256-271 set forth in SEQ ID NOs 2 and 10, respectively;
(ii) FS22-172-003-AA/FS28-024-052 set forth in SEQ ID NOs 18 and 26, respectively;
(iii) FS22-172-003-AA/FS28-256-021 set forth in SEQ ID NOs 30 and 32, respectively;
(iv) FS22-172-003-AA/FS28-256-012 set forth in SEQ ID NOs 36 and 37, respectively;
(v) FS22-172-003-AA/FS28-256-023 set forth in SEQ ID NOs 41 and 32, respectively;
(vi) FS22-172-003-AA/FS28-256-024 set forth in SEQ ID NOs 30 and 47, respectively;
(vii) FS22-172-003-AA/FS28-256-026 set forth in SEQ ID NOs 41 and 47, respectively;
(viii) FS22-172-003-AA/FS28-256-027 set forth in SEQ ID NOs 30 and 10, respectively;
(ix) FS22-172-003-AA/FS28-256-001 set forth in SEQ ID NOs 51 and 32, respectively;
(x) FS22-172-003-AA/FS28-256-005 set forth in SEQ ID NOs 51 and 47, respectively;
(xi) FS22-172-003-AA/FS28-256-014 set forth in SEQ ID NOs 58 and 37, respectively;
(xii) FS22-172-003-AA/FS28-256-018 set forth in SEQ ID NOs 41 and 37, respectively;
(xiii) FS22-172-003-AA/FS28-256 set forth in SEQ ID NOs 65 and 37, respectively;
(xiv) FS22-172-003-AA/FS28-024-051 set forth in SEQ ID NOs 70 and 26, respectively;
(xv) FS22-172-003-AA/FS28-024-053 set forth in SEQ ID NOs 75 and 26, respectively; or
(xvi) FS22-172-003-AA/FS28-024 set forth in SEQ ID NOs 80 and 26, respectively.


SEQUENCE LISTING (92 sequences)

SEQ ID NO: 1 - protein, 453 aa, synthetic construct
  REGION 1..453: note = FS22-172-003-AA/FS28-256-271 mAb2: Heavy chain AA (without LALA)
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 2 - protein, 453 aa, synthetic construct
  REGION 1..453: note = Heavy chain AA (with LALA)
EVQLLESGGGLVQPGGSLRLSCAASGFTFTHTYMSWVRQAPGKGLEWVSAISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 3 - protein, 119 aa, synthetic construct
  REGION 1..119: note = VH domain AA
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSS

SEQ ID NO: 4 - protein, 7 aa, synthetic construct
  REGION 1..7: note = LCDR1 (AA) (IMGT)
QSVSSSY

SEQ ID NO: 5 - protein, 5 aa, synthetic construct
  REGION 1..5: note = HCDR1 (AA) (Kabat)
HTYMS

SEQ ID NO: 6 - protein, 8 aa, synthetic construct
  REGION 1..8: note = HCDR2 (AA) (IMGT)
ISPTYSTT

SEQ ID NO: 7 - protein, 17 aa, synthetic construct
  REGION 1..17: note = HCDR2 (AA) Kabat)
AISPTYSTTNYADSVKG

SEQ ID NO: 8 - protein, 12 aa, synthetic construct
  REGION 1..12: note = HCDR3 (AA) (IMGT)
ARYNAYHAALDY

SEQ ID NO: 9 - protein, 10 aa, synthetic construct
  REGION 1..10: note = HCDR3 (AA) (Kabat)
YNAYHAALDY

SEQ ID NO: 10 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQTVPYPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 11 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQTVPYPYTFGQGTKVEIK

SEQ ID NO: 12 - protein, 7 aa, synthetic construct
  REGION 1..7: note = LCDR1 (AA) (IMGT)
QSVSSSY

SEQ ID NO: 13 - protein, 12 aa, synthetic construct
  REGION 1..12: note = LCDR1 (AA) (Kabat)
RASQSVSSSYLA

SEQ ID NO: 14 - protein, 4 aa, synthetic construct
  REGION 1..4: note = LCDR2 (AA) (IMGT)
GASG

SEQ ID NO: 15 - protein, 7 aa, synthetic construct
  REGION 1..7: note = LCDR2 (AA) (Kabat)
GASSRAT

SEQ ID NO: 16 - protein, 9 aa, synthetic construct
  REGION 1..9: note = LCDR3 (AA) (IMGT) and (Kabat)
QQTVPYPYT

SEQ ID NO: 17 - protein, 450 aa, synthetic construct
  REGION 1..450: note = FS22-172-003-AA/FS28-024-052 mAb2: Heavy chain AA (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 18 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 19 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALLFDYWGQGTLVTVSS

SEQ ID NO: 20 - protein, 8 aa, synthetic construct
  REGION 1..8: note = HCDR1 (AA) (IMGT)
GFTLSYSS

SEQ ID NO: 21 - protein, 5 aa, synthetic construct
  REGION 1..5: note = HCDR1 (AA) (Kabat)
YSSMS

SEQ ID NO: 22 - protein, 8 aa, synthetic construct
  REGION 1..8: note = HCDR2 (AA) (IMGT)
ITPSTGYT

SEQ ID NO: 23 - protein, 17 aa, synthetic construct
  REGION 1..17: note = HCDR2 (AA) Kabat)
FITPSTGYTHYADSVKG

SEQ ID NO: 24 - protein, 9 aa, synthetic construct
  REGION 1..9: note = HCDR3 (AA) (IMGT)
ARRALLFDY

SEQ ID NO: 25 - protein, 7 aa, synthetic construct
  REGION 1..7: note = HCDR3 (AA) (Kabat)
RALLFDY

SEQ ID NO: 26 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQASSYPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQASSYPLTFGQGTKVEIK

SEQ ID NO: 28 - protein, 9 aa, synthetic construct
  REGION 1..9: note = LCDR3 (AA) (IMGT) and (Kabat)
QQASSYPLT

SEQ ID NO: 29 - protein, 453 aa, synthetic construct
  REGION 1..453: note = FS22-172-003-AA/FS28-256-021 mAb2: Heavy chain AA (without LALA)
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 30 - protein, 453 aa, synthetic construct
  REGION 1..453: note = Heavy chain AA (with LALA)
EVQLLESGGGLVQPGGSLRLSCAASGFTFTHTYMSWVRQAPGKGLEWVSNISPTYSTTNYADSVKGRFTISRDNNKNTLYLQMNSLRAEDTAVYYCARYNAYHAALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 31 - protein, 17 aa, synthetic construct
  REGION 1..17: note = HCDR2 (AA) Kabat)
NISPTYSTTNYADSVKG

SEQ ID NO: 32 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQHNQYPNTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQHNQYPNTFGQGTKVEIK

SEQ ID NO: 34 - protein, 9 aa, synthetic construct
  REGION 1..9: note = LCDR3 (AA) (IMGT) and (Kabat)
QQHNQYPNT

SEQ ID NO: 35 - protein, 453 aa, synthetic construct; identical to SEQ ID NO: 29
  REGION 1..453: note = FS22-172-003-AA/FS28-256-012 mAb2: Heavy chain AA (without LALA)

SEQ ID NO: 36 - protein, 453 aa, synthetic construct; identical to SEQ ID NO: 30
  REGION 1..453: note = Heavy chain AA (with LALA)

SEQ ID NO: 37 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQSYYYPITFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQSYYYPITFGQGTKVEIK

SEQ ID NO: 39 - protein, 9 aa, synthetic construct
  REGION 1..9: note = LCDR3 (AA) (IMGT) and (Kabat)
QQSYYYPIT

SEQ ID NO: 40 - protein, 453 aa, synthetic construct
  REGION 1..453: note = FS22-172-003-AA/FS28-256-023 mAb2: Heavy chain AA (without LALA)
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 41 - protein, 354 aa, synthetic construct
  REGION 1..354: note = Heavy chain AA (with LALA)
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLP

SEQ ID NO: 42 - protein, 119 aa, synthetic construct
  REGION 1..119: note = VH domain AA
EVQLLESGGGLVQPGGSLRLSCAASGFTFTQTYMSWVRQAPGKGLEWVSNISPTYSTTNYADSVKGRFTISRDNSKNTLYLQMNSLRAEDTAVYYCARYNAYQIGLDYWGQGTLVTVSS

SEQ ID NO: 43 - protein, 8 aa, synthetic construct
  REGION 1..8: note = HCDR1 (AA) (IMGT)
GFTFTQTY

SEQ ID NO: 44 - protein, 5 aa, synthetic construct
  REGION 1..5: note = HCDR1 (AA) (Kabat)
QTYMS

SEQ ID NO: 45 - protein, 12 aa, synthetic construct
  REGION 1..12: note = HCDR3 (AA) (IMGT)
ARYNAYQIGLDY

SEQ ID NO: 46 - protein, 10 aa, synthetic construct
  REGION 1..10: note = HCDR3 (AA) (Kabat)
YNAYQIGLDY

SEQ ID NO: 47 - protein, 215 aa, synthetic construct
  REGION 1..215: note = FS22-172-003-AA/FS28-256-024 mAb2: Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQALGYPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 108 aa, synthetic construct
  REGION 1..108: note = VL domain AA
EIVLTQSPGTLSLSPGERATLSCRASQSVSSSYLAWYQQKPGQAPRLLIYGASSRATGIPDRFSGSGSGTDFTLTISRLEPEDFAVYYCQQALGYPHTFGQGTKVEIK

SEQ ID NO: 49 - protein, 9 aa, synthetic construct
  REGION 1..9: note = LCDR3 (AA) (IMGT) and (Kabat)
QQALGYPHT

SEQ ID NO: 50 - protein, 453 aa, synthetic construct
  REGION 1..453: note = FS22-172-003-AA/FS28-256-001 mAb2: Heavy chain AA (without LALA)
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 51 - protein, 453 aa, synthetic construct
  REGION 1..453: note = Heavy chain AA (with LALA)
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 52 - protein, 119 aa, synthetic construct
  REGION 1..119: note = VH domain AA
EVQLLESGGGLVQPGGSLRLSCAASGFTFTETYMSWVRQAPGKGLEWVSNISPTYSTTNYADSVKGRFTISRDNSKNTLYLQMNSLRAEDTAVYYCARYNSYQGGLDYWGQGTLVTVSS

SEQ ID NO: 53 - protein, 8 aa, synthetic construct
  REGION 1..8: note = HCDR1 (AA) (IMGT)
GFTFTETY

SEQ ID NO: 54 - protein, 5 aa, synthetic construct
  REGION 1..5: note = HCDR1 (AA) (Kabat)
ETYMS

SEQ ID NO: 55 - protein, 12 aa, synthetic construct
  REGION 1..12: note = HCDR3 (AA) (IMGT)
ARYNSYQGGLDY

SEQ ID NO: 56 - protein, 10 aa, synthetic construct
  REGION 1..10: note = HCDR3 (AA) (Kabat)
YNSYQGGLDY

SEQ ID NO: 57 - protein, 453 aa, synthetic construct
  REGION 1..453: note = FS22-172-003-AA/FS28-256-014 mAb2: Heavy chain AA (without LALA)
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 58 - protein, 453 aa, synthetic construct
  REGION 1..453: note = Heavy chain AA (with LALA)
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 59 - protein, 119 aa, synthetic construct
  REGION 1..119: note = VH domain AA
EVQLLESGGGLVQPGGSLRLSCAASGFTFTDTYMSWVRQAPGKGLEWVSNISPTYSTTNYADSVKGRFTISRDNSKNTLYLQMNSLRAEDTAVYYCARYNAYAAGLDYWGQGTLVTVSS

SEQ ID NO: 60 - protein, 8 aa, synthetic construct
  REGION 1..8: note = HCDR1 (AA) (IMGT)
GFTFTDTY

SEQ ID NO: 61 - protein, 5 aa, synthetic construct
  REGION 1..5: note = HCDR1 (AA) (Kabat)
DTYMS

SEQ ID NO: 62 - protein, 12 aa, synthetic construct
  REGION 1..12: note = HCDR3 (AA) (IMGT)
ARYNAYAAGLDY

SEQ ID NO: 63 - protein, 10 aa, synthetic construct
  REGION 1..10: note = HCDR3 (AA) (Kabat)
YNAYAAGLDY

SEQ ID NO: 64 - protein, 453 aa, synthetic construct
  REGION 1..453: note = of FS22-172-003-AA/FS28-256 mAb2: Heavy chain AA (without LALA)
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 65 - protein, 453 aa, synthetic construct
  REGION 1..453: note = Heavy chain AA (with LALA)
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 66 - protein, 119 aa, synthetic construct
  REGION 1..119: note = VH domain AA
EVQLLESGGGLVQPGGSLRLSCAASGFTFTNTYMSWVRQAPGKGLEWVSNISPTYSTTNYADSVKGRFTISRDNSKNTLYLQMNSLRAEDTAVYYCARYNSYQGGLDYWGQGTLVTVSS

SEQ ID NO: 67 - protein, 8 aa, synthetic construct
  REGION 1..8: note = HCDR1 (AA) (IMGT)
GFTFTNTY

SEQ ID NO: 68 - protein, 5 aa, synthetic construct
  REGION 1..5: note = HCDR1 (AA) (Kabat)
NTYMS

SEQ ID NO: 69 - protein, 450 aa, synthetic construct
  REGION 1..450: note = FS22-172-003-AA/FS28-024-051 mAb2: Heavy chain AA (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 70 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 71 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALIFDYWGQGTLVTVSS

SEQ ID NO: 72 - protein, 9 aa, synthetic construct
  REGION 1..9: note = HCDR3 (AA) (IMGT)
ARRALIFDY

SEQ ID NO: 73 - protein, 7 aa, synthetic construct
  REGION 1..7: note = HCDR3 (AA) (Kabat)
RALIFDY

SEQ ID NO: 74 - protein, 450 aa, synthetic construct
  REGION 1..450: note = FS22-172-003-AA/FS28-024-053 mAb2: Heavy chain AA (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 75 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 76 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALVFDYWGQGTLVTVSS

SEQ ID NO: 77 - protein, 9 aa, synthetic construct
  REGION 1..9: note = HCDR3 (AA) (IMGT)
ARRALVFDY

SEQ ID NO: 78 - protein, 7 aa, synthetic construct
  REGION 1..7: note = HCDR3 (AA) (Kabat)
RALVFDY

SEQ ID NO: 79 - protein, 450 aa, synthetic construct
  REGION 1..450: note = FS22-172-003-AA/FS28-024 mAb2: Heavy chain AA (without LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 80 - protein, 450 aa, synthetic construct
  REGION 1..450: note = Heavy chain AA (with LALA)
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 81 - protein, 116 aa, synthetic construct
  REGION 1..116: note = VH domain AA
EVQLLESGGGLVQPGGSLRLSCAASGFTLSYSSMSWVRQAPGKGLEWVSFITPSTGYTHYADSVKGRFTISRDNSKNTLYLQMNSLRAEDTAVYYCARRALTFDYWGQGTLVTVSS

SEQ ID NO: 82 - protein, 9 aa, synthetic construct
  REGION 1..9: note = HCDR3 (AA) (IMGT)
ARRALTFDY

SEQ ID NO: 83 - protein, 7 aa, synthetic construct
  REGION 1..7: note = HCDR3 (AA) (Kabat)
RALTFDY

SEQ ID NO: 84 - protein, 448 aa, synthetic construct
  REGION 1..448: note = FS122m (surrogate anti-mMSLN mCD137 Fcab G1-AA): Heavy chain AA (with LALA)
EVQLLESGGGLVQPGGSLRLSCAASGFTFSTYFMVWVRQAPGKGLEWVSMISPKSSNTYYADSVKGRFTISRDNSKNTLYLQMNSLRAEDTAVYYCARYHISPRFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDEPYWSYVSLTCLVKGFYPSDIAVEWESNGQPENNYKTTPPVLDSDGSFFLYSKLTVMNYRWELGNVFSCSVMHEALHNHYTQKSLSLSPGK

SEQ ID NO: 85 - protein, 215 aa, synthetic construct
  REGION 1..215: note = Light chain AA
EIVLTQSPGTLSLSPGERATLSCRASQSVSSSYLAWYQQKPGQAPRLLIYGASSRATGIPDRFSGSGSGTDFTLTISRLEPEDFAVYYCQQPFPFSFTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 86 - protein, 111 aa, synthetic construct
  REGION 1..111: note = CH3 AB and EF structural loops of all FS22-172-003 Fcab-containing mAb2 clones and the FS22-172-003 Fcab: CH3 domain
GQPREPQVYTLPPSRDELPYIIPPYNQVSLTCLVKGFYPSDIAVEWESNGQPENNYKTTPPVLDSDGSFFLYSKLTVGADRWLEGNVFSCSVMHEALHNHYTQKSLSLSPG

SEQ ID NO: 87 - protein, 7 aa, synthetic construct
  REGION 1..7: note = AB Loop
PYIIPPY

SEQ ID NO: 88 - protein, 7 aa, synthetic construct
  REGION 1..7: note = EF Loop
GADRWLE

SEQ ID NO: 89 - protein, 9 aa, synthetic construct
  REGION 1..9: note = CD Loop
SNGQPENNY

SEQ ID NO: 90 - protein, 110 aa, synthetic construct
  REGION 1..110: note = CH2 domain containing LALA mutation, PA mutation or LALA-PA mutation: CH2 (LALA)
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 91 - protein, 110 aa, synthetic construct
  REGION 1..110: note = CH2 (PA)
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK

SEQ ID NO: 92 - protein, 110 aa, synthetic construct
  REGION 1..110: note = CH2 (LALA-PA)
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAK